(19) United States
(12) Patent Application Publication
(10) Pub. No.: US 2008/0064727 A1
(43) Pub. Date: Mar. 13, 2008

(54) Crystalline forms of tiagabine hydrochloride

(75) Inventors: Scott L. Childs, Atlanta, GA (US); Leonard J. Chyall, Lafayette, IN (US); Karen S. Gushurst, West Lafayette, IN (US); R. Curtis Haltiwanger, West Chester, PA (US); Robert E. McKean, Chester Springs, PA (US); Donglai Yang, West Lafayette, IN (US)

Correspondence Address: CEPHALON, INC., 41 MOORES ROAD, PO BOX 4011, FRAZER, PA, 19355, US

(73) Assignee: Cephalon, Inc., Frazer, PA (US)

(21) Appl. No.: 11/893,618

(22) Filed: Aug. 16, 2007

Related U.S. Application Data
(60) Provisional application No. 60/838,763, filed on Aug. 18, 2006.

(51) Int. Cl. A61K 31/496; A61P 25/00; C07D 409/14

(52) U.S. Cl. 514/326; 546/212

(57) ABSTRACT

The present invention provides 16 new crystalline forms of tiagabine hydrochloride.

### BACKGROUND OF THE INVENTION

1. Technical Field

This invention relates to crystalline forms of tiagabine hydrochloride.

2. Background Art

Tiagabine ((−)-(R)-1-(4,4-bis(3-methyl-2-thienyl)-3-butenyl)-3-piperidinecarboxylic acid; CAS # 115103-54-3) is a gamma-aminobutyric acid (GABA) uptake inhibitor. Tiagabine is often used as an adjunctive therapy in adults and children twelve (12) years and older for treatment of partial seizures, and is marketed in the form of its hydrochloride salt under the trade name GABITRIL® (Cephalon, Inc., Frazer, Pa.). Tiagabine hydrochloride has the following chemical structure:

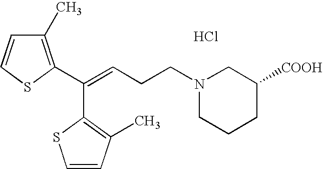

U.S. Pat. No. 5,010,090 (the '090 patent) discloses crystalline tiagabine hydrochloride prepared by crystallization from ethyl acetate, isopropanol, acetone, or water. The '090 patent does not disclose the x-ray diffraction pattern, solvent content, differential scanning calorimetry (DSC) pattern, thermogravimetric analysis (TGA), or nuclear magnetic resonance (NMR) spectrum of the prepared tiagabine hydrochloride.

U.S. Pat. No. 5,354,760 (the '760 patent) discloses a monohydrate crystalline form of tiagabine hydrochloride. This crystalline form is referred to herein as tiagabine hydrochloride monohydrate or tiagabine hydrochloride Form A. The '760 patent discloses the preparation of tiagabine hydrochloride Form A by crystallizing tiagabine hydrochloride from water or aqueous hydrochloric acid. The '760 patent provides X-ray powder diffraction (XRPD), ¹H-NMR, infrared (IR) spectroscopy, DSC, and water content characterization data for the obtained crystalline form. The '760 patent states that crystallizing tiagabine hydrochloride from solvents such as ethyl acetate, acetonitrile, butyl acetate, toluene, acetone, or dichloromethane gives products containing varying amounts of the used crystallizing solvent, but no organic solvent solvate crystalline form of tiagabine hydrochloride is disclosed.

U.S. Pat. No. 5,958,951 (the '951 patent) discloses an anhydrous crystalline form of tiagabine hydrochloride. This crystalline form is referred to herein as tiagabine hydrochloride anhydrous or tiagabine hydrochloride Form B. The '951 patent discloses the preparation of tiagabine hydrochloride Form B by crystallizing tiagabine hydrochloride from aqueous hydrochloric acid under specified conditions. The '951 patent provides XRPD, DSC, TGA, and water content characterization data for tiagabine hydrochloride Form B. The '951 patent states that crystallizing tiagabine hydrochloride from ethyl acetate gives products containing unwanted amounts of the crystallizing solvent; and the use of other organic solvents often results in the formation of solvates of tiagabine hydrochloride, but no organic solvent solvate crystalline form of tiagabine hydrochloride is disclosed.

WO 2005/092886 A1 (the '886 application) discloses an amorphous form of tiagabine hydrochloride prepared by spray drying a methanol solution of tiagabine hydrochloride. XRPD, IR, and DSC data are provided. No crystalline form is disclosed.

There is a continuing need for additional crystalline forms of tiagabine hydrochloride.

### SUMMARY OF THE INVENTION

The present invention provides a crystalline form of tiagabine hydrochloride chosen from Forms C, D, H, I, J, M, P, Q, T, W, Y, Z, AA, S, X, and AB. Preferably, the crystalline form exhibits an x-ray powder diffraction pattern having characteristic peaks as set forth in the following Table 1:

TABLE 1
Characteristic XRPD Peaks of Tiagabine HCl Crystalline Forms

| Form | Characteristic XRPD Peaks (±0.2 degrees 2θ) | | | | | | | | | |
|---|---|---|---|---|---|---|---|---|---|---|
| C | 6.1 | 7.9 | 8.7 | 12.7 | 14.8 | 16.1 | 17.2 | 22.9 | 25.1 | 25.9 |
| D | 7.9 | 12.7 | 14.4 | 16.9 | 17.1 | 18.1 | 18.8 | 21.5 | 22.0 | 24.3 |
| H | 5.8 | 7.6 | 7.8 | 11.6 | 14.6 | 15.9 | 17.0 | 19.7 | 22.6 | 25.1 |
| I | 10.5 | 12.5 | 13.1 | 15.0 | 17.3 | 20.6 | 21.0 | 24.8 | 25.2 | 27.0 |
| J | 7.8 | 12.4 | 13.0 | 14.6 | 17.0 | 17.5 | 21.1 | 21.8 | 24.8 | 26.2 |
| M | 7.8 | 12.8 | 14.5 | 16.9 | 21.1 | 21.8 | 24.5 | 24.9 | 26.3 | 27.5 |
| P | 12.5 | 14.5 | 16.1 | 17.6 | 21.9 | 25.2 | 26.5 | 35.8 | 37.7 | 39.3 |
| Q | 6.4 | 11.4 | 12.9 | 14.8 | 15.3 | 16.7 | 18.8 | 22.9 | 24.7 | 25.3 |
| T | 7.9 | 8.6 | 12.6 | 15.9 | 17.1 | 18.3 | 20.8 | 22.2 | 23.5 | 25.0 |
| W | 12.6 | 13.2 | 16.6 | 17.0 | 17.6 | 18.6 | 21.0 | 23.9 | 24.3 | 24.8 |
| Y | 7.7 | 11.6 | 14.6 | 16.7 | 16.9 | 18.6 | 18.9 | 21.4 | 22.4 | 25.6 |
| Z | 5.6 | 8.3 | 11.4 | 11.7 | 13.2 | 16.4 | 16.9 | 19.9 | 20.7 | 23.9 |
| AA | 7.4 | 11.2 | 13.1 | 14.7 | 16.6 | 18.2 | 20.0 | 22.0 | 22.4 | 24.0 |
| S | 6.7 | 7.9 | 12.5 | 13.1 | 17.6 | 21.8 | 27.7 | — | — | — |
| X | 7.8 | 11.7 | 14.0 | 15.6 | 18.5 | 18.9 | 24.9 | — | — | — |
| AB | 4.1 | 7.6 | 14.0 | 17.8 | 18.4 | — | — | — | — | — |

Preferably, the crystalline form is chosen from Forms C, Q, W and AA. Preferably, the crystalline form has a purity of at least about 50% (w/w).

The present invention further provides a pharmaceutical composition comprising one or more of the above crystalline forms of tiagabine hydrochloride and one or more pharmaceutically acceptable excipients.

The present invention further provides a process for preparing a crystalline form of tiagabine hydrochloride comprising the steps of:
(a) crystallizing tiagabine hydrochloride from isopropanol to provide tiagabine hydrochloride Form C; or
(b) crystallizing tiagabine hydrochloride from acetonitrile to provide tiagabine hydrochloride Form D; or
(c) crystallizing tiagabine hydrochloride from methyl ethyl ketone to provide tiagabine hydrochloride Form H; or
(d) crystallizing tiagabine hydrochloride from acetone to provide tiagabine hydrochloride Form I; or
(e) crystallizing tiagabine hydrochloride from ethanol to provide tiagabine hydrochloride Form J; or
(f) crystallizing tiagabine hydrochloride from dichloromethane to provide tiagabine hydrochloride Form M; or
(g) crystallizing tiagabine hydrochloride from a solvent selected from 1,4-dioxane and methyl ethyl ketone to provide tiagabine hydrochloride Form P; or
(h) crystallizing tiagabine hydrochloride from methyl t-butyl ether to provide tiagabine hydrochloride Form Q; or
(i) drying tiagabine hydrochloride Form H in a vacuum oven to provide tiagabine hydrochloride Form Q; or
(j) crystallizing tiagabine hydrochloride from 2-butanol to provide tiagabine hydrochloride Form T; or
(k) crystallizing tiagabine hydrochloride from acetone to provide tiagabine hydrochloride Form W; or
(l) crystallizing tiagabine hydrochloride from a mixture of acetone and cyclohexane to provide tiagabine hydrochloride Form W; or
(m) crystallizing tiagabine hydrochloride from 1,4-dioxane to provide tiagabine hydrochloride Form Y; or
(n) crystallizing tiagabine hydrochloride from tetrahydrofuran to provide tiagabine hydrochloride Form Z; or
(o) slurrying tiagabine hydrochloride monohydrate in acetone to provide tiagabine hydrochloride Form AA; or
(p) storing tiagabine hydrochloride Form I at room temperature for about two (2) months to provide tiagabine hydrochloride Form S; or
(q) crystallizing tiagabine hydrochloride from water to provide tiagabine hydrochloride Form X; or
(r) heating tiagabine hydrochloride monohydrate at 150° C. to provide tiagabine hydrochloride Form AB.

The present invention further provides a process for preparing amorphous tiagabine hydrochloride, comprising the steps of:
(a) heating tiagabine hydrochloride at or above its melting point, and
(b) cooling the heated tiagabine hydrochloride.
   Preferably, the cooling step (b) is performed by immersing a container of the melted tiagabine hydrochloride in an ice bath. Preferably, the cooling step (b) is performed by immersing a container of the melted tiagabine hydrochloride in a dry ice/isopropanol bath.

The present invention further provides a process for preparing amorphous tiagabine hydrochloride, comprising the steps of:
(a) preparing an aqueous solution of tiagabine hydrochloride, and
(b) freeze drying the aqueous solution of tiagabine hydrochloride.

### BRIEF DESCRIPTION OF THE DIAGRAMS

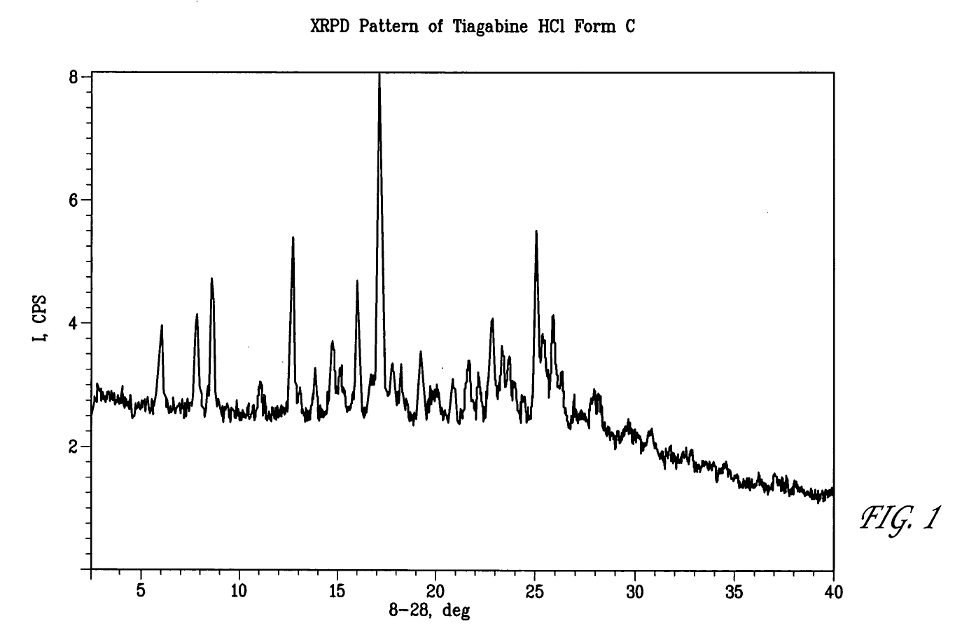

FIG. 1 depicts an x-ray powder diffraction (XRPD) pattern of tiagabine hydrochloride Form C.

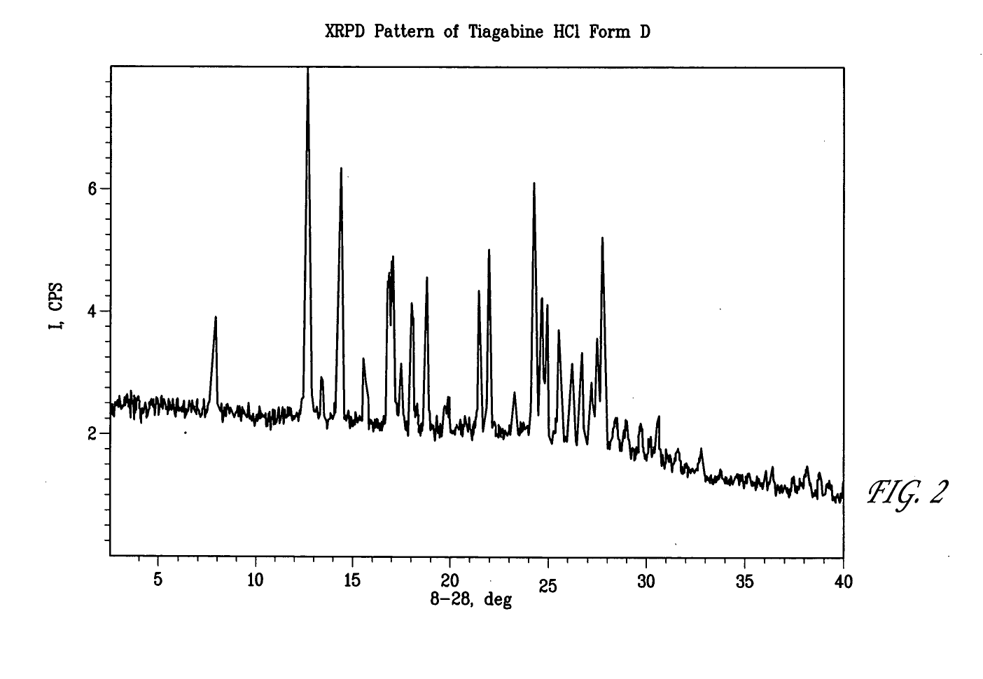

FIG. 2 depicts an XRPD pattern of tiagabine hydrochloride Form D.

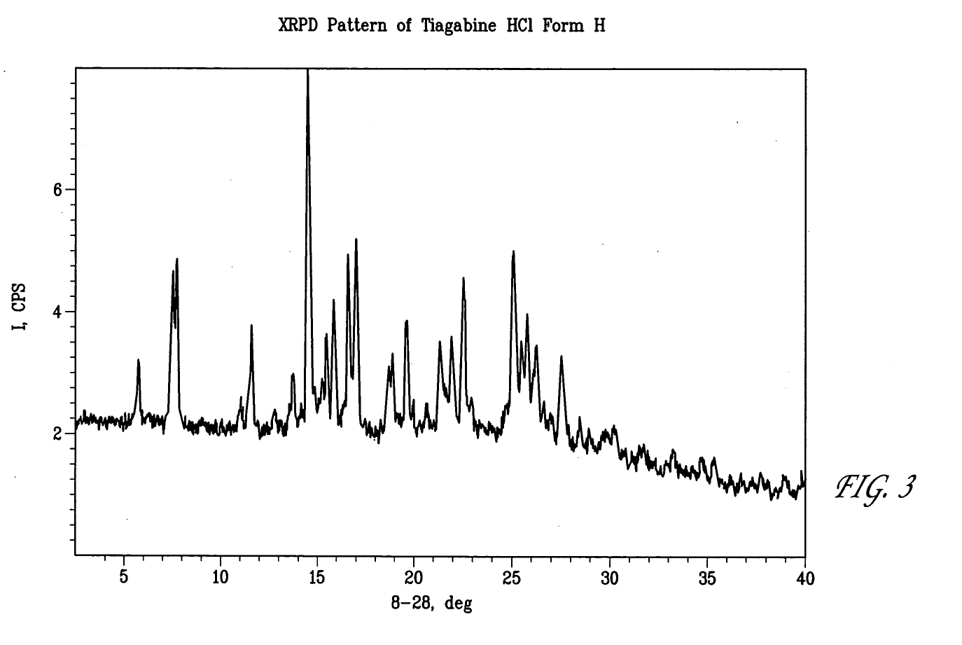

FIG. 3 depicts an XRPD pattern of tiagabine hydrochloride Form H.

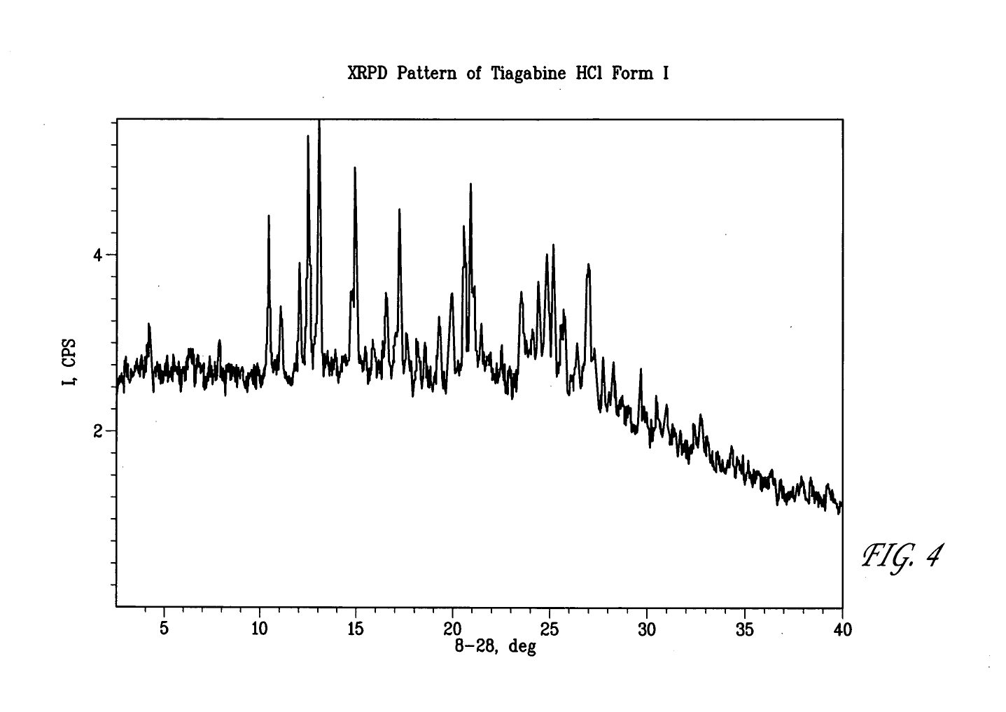

FIG. 4 depicts an XRPD pattern of tiagabine hydrochloride Form I.

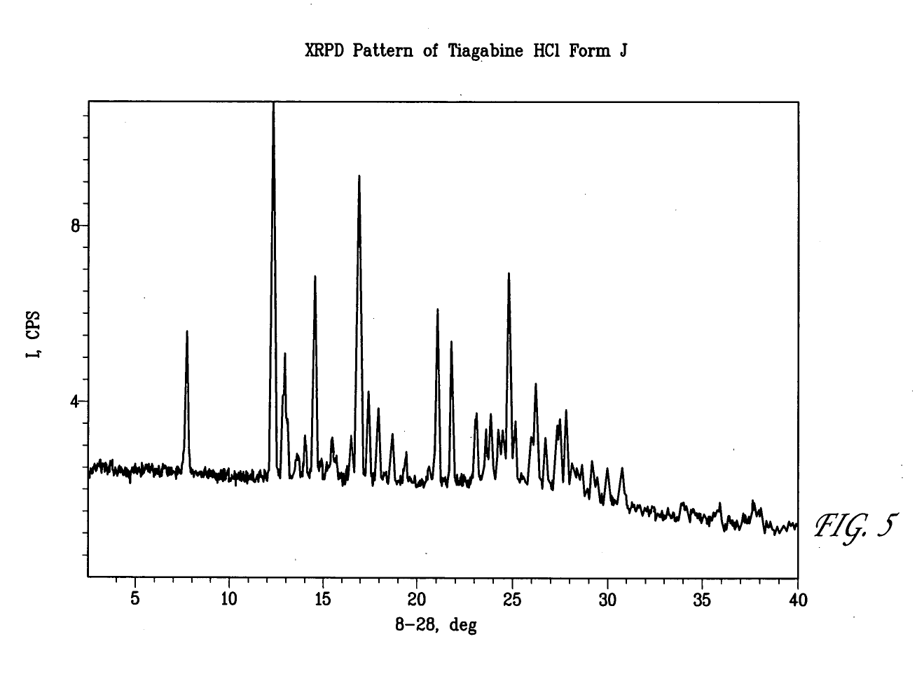

FIG. 5 depicts an XRPD pattern of tiagabine hydrochloride Form J.

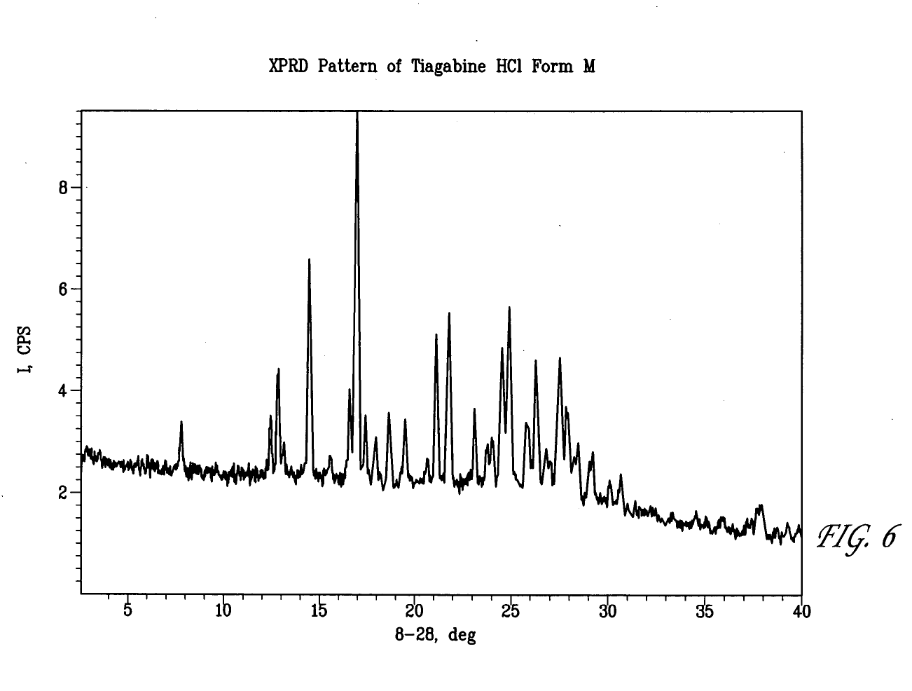

FIG. 6 depicts an XRPD pattern of tiagabine hydrochloride Form M.

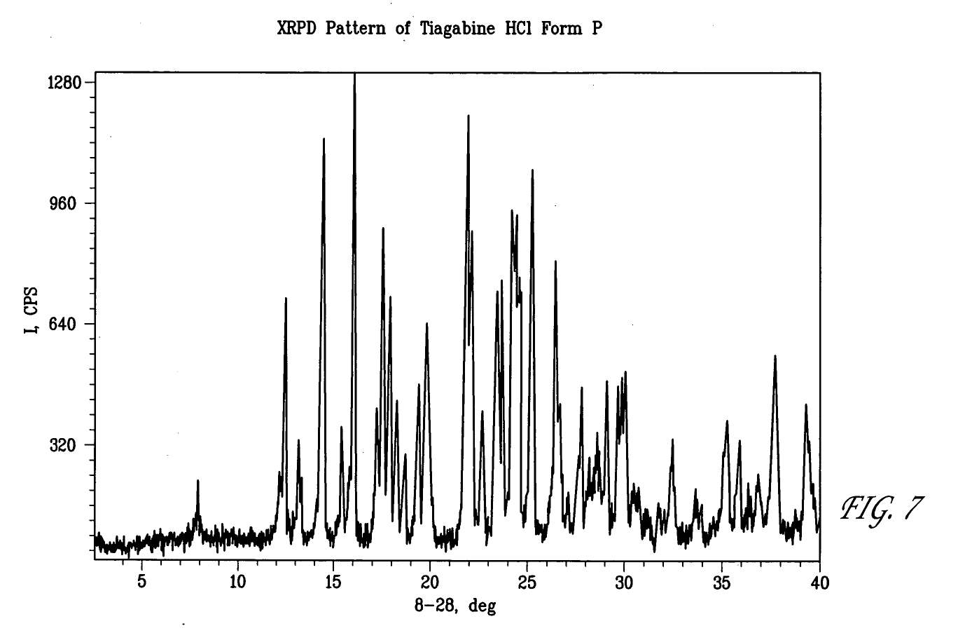

FIG. 7 depicts an XRPD pattern of tiagabine hydrochloride Form P.

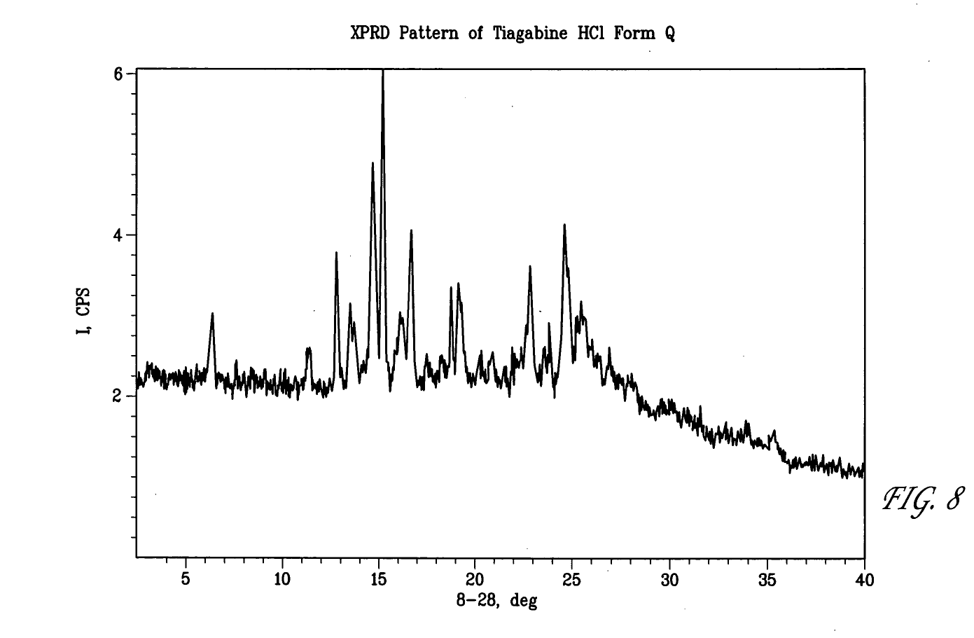

FIG. 8 depicts an XRPD pattern of tiagabine hydrochloride Form Q.

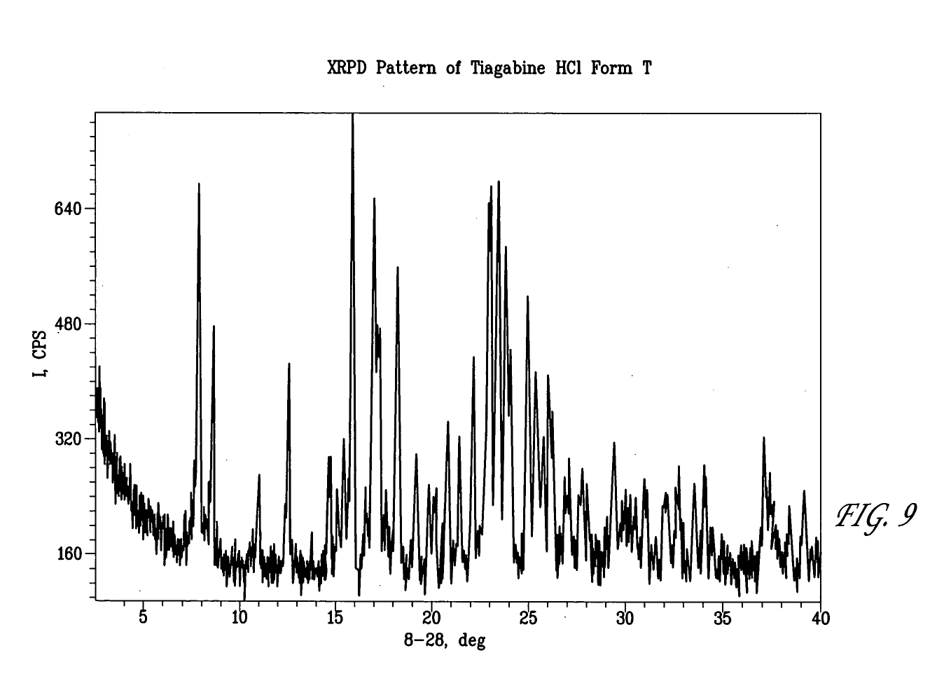

FIG. 9 depicts an XRPD pattern of tiagabine hydrochloride Form T.

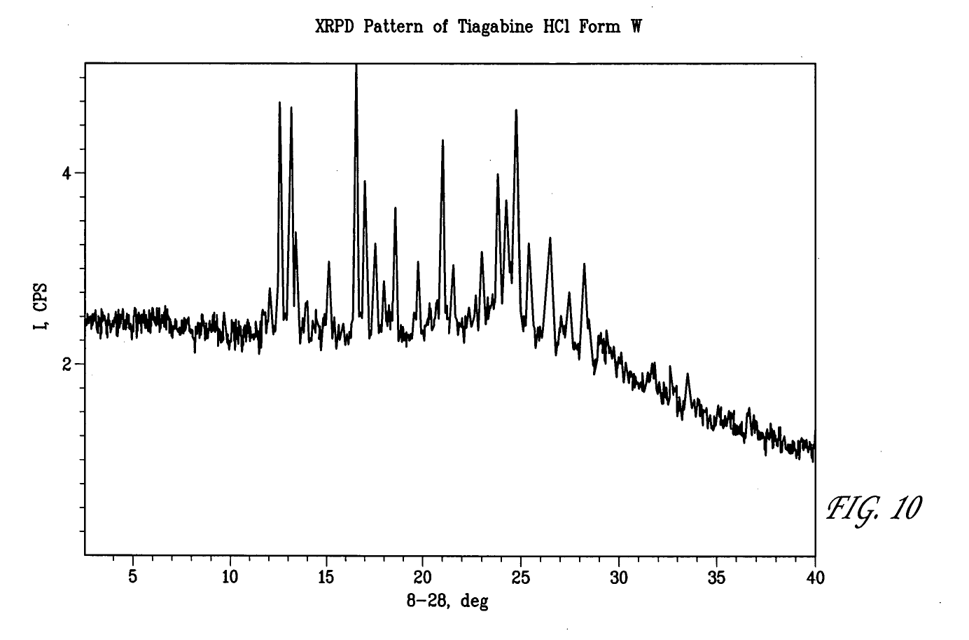

FIG. 10 depicts an XRPD pattern of tiagabine hydrochloride Form W.

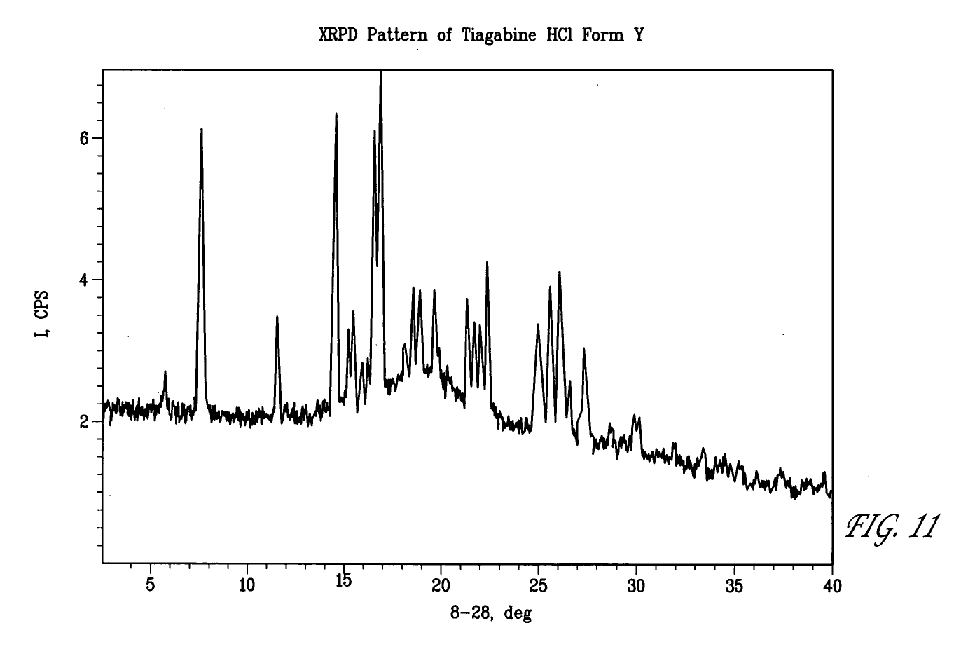

FIG. 11 depicts an XRPD pattern of tiagabine hydrochloride Form Y.

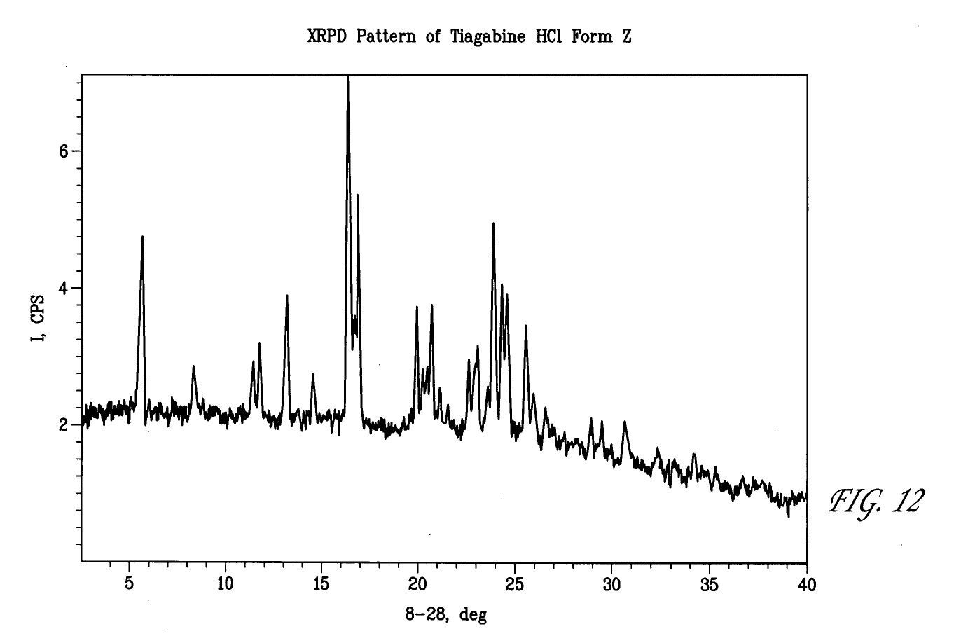

FIG. 12 depicts an XRPD pattern of tiagabine hydrochloride Form Z.

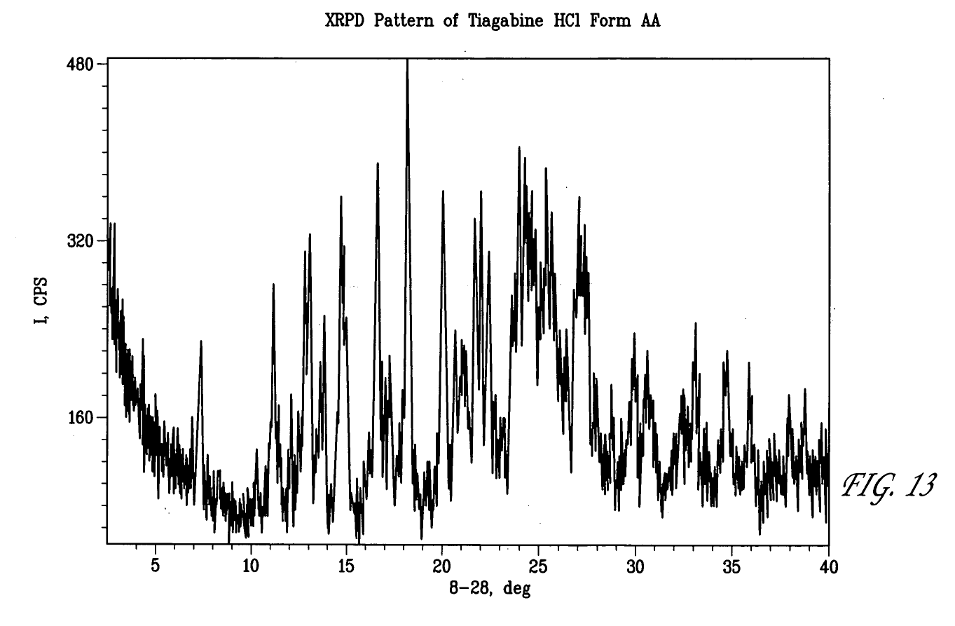

FIG. 13 depicts an XRPD pattern of tiagabine hydrochloride Form AA.

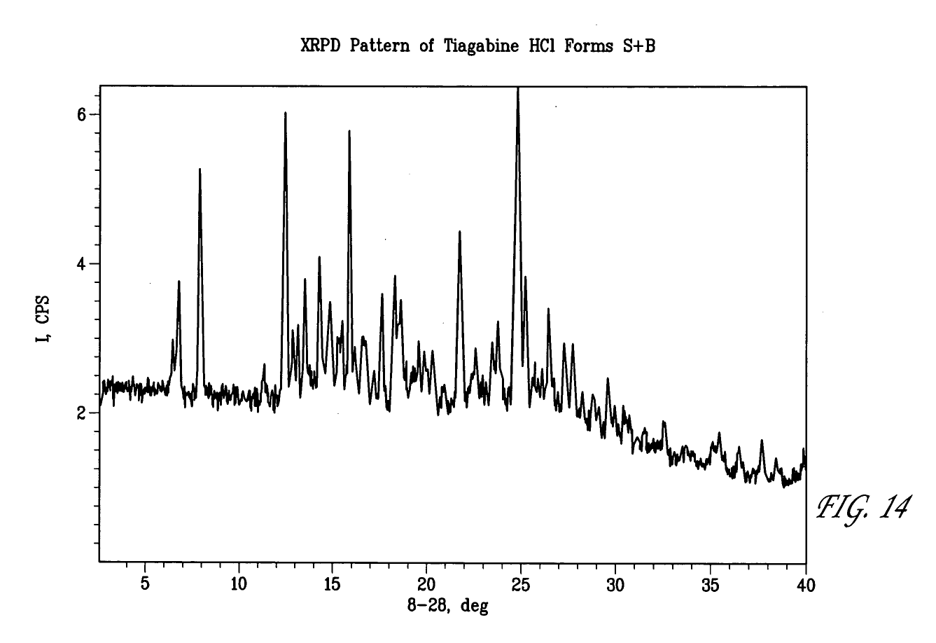

FIG. 14 depicts an XRPD pattern of tiagabine hydrochloride Form S+B.

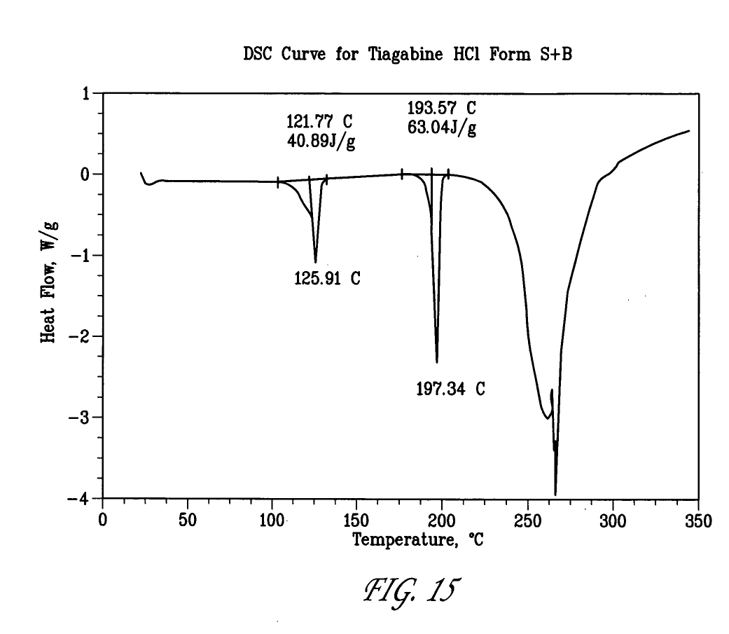

FIG. 15 depicts a differential scanning calorimetry (DSC) curve of tiagabine hydrochloride Form S+B.

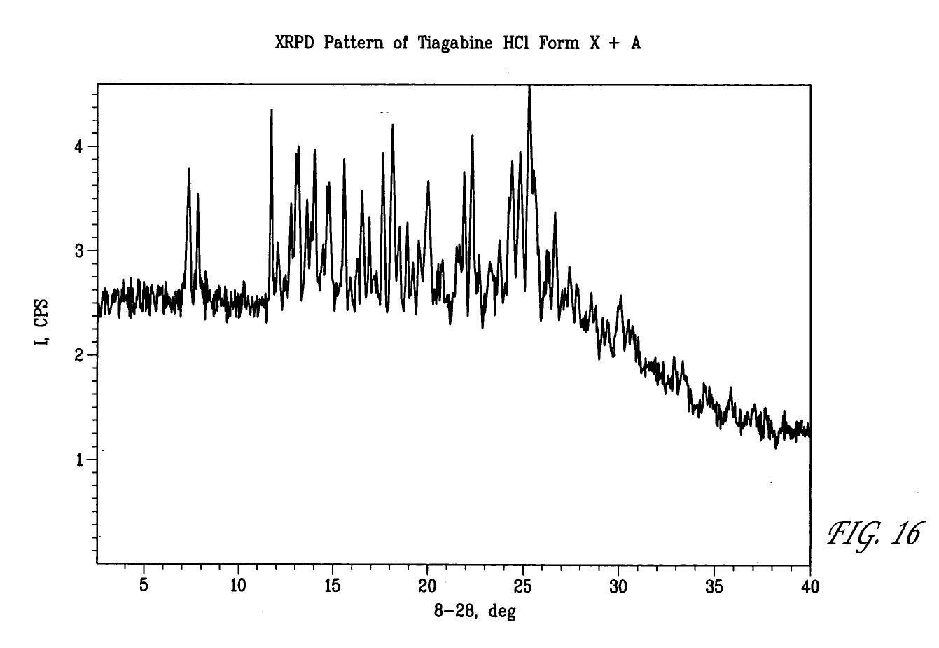

FIG. 16 depicts an XRPD pattern of tiagabine hydrochloride Form X+A.

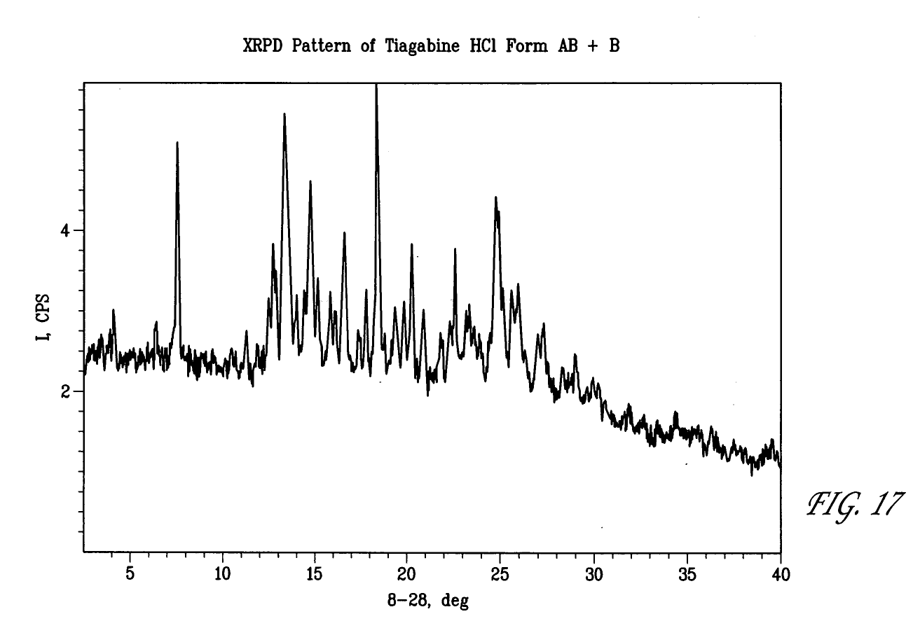

FIG. 17 depicts an XRPD pattern of tiagabine hydrochloride Form AB+B.

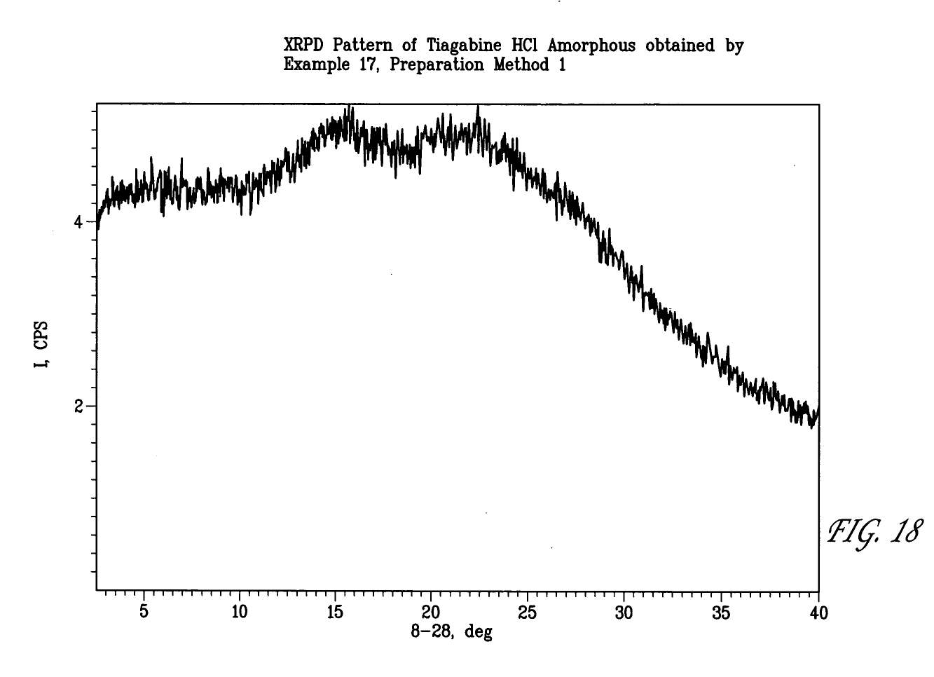

FIG. 18 depicts an XRPD pattern of tiagabine hydrochloride amorphous obtained by Example 17, Preparation Method 1.

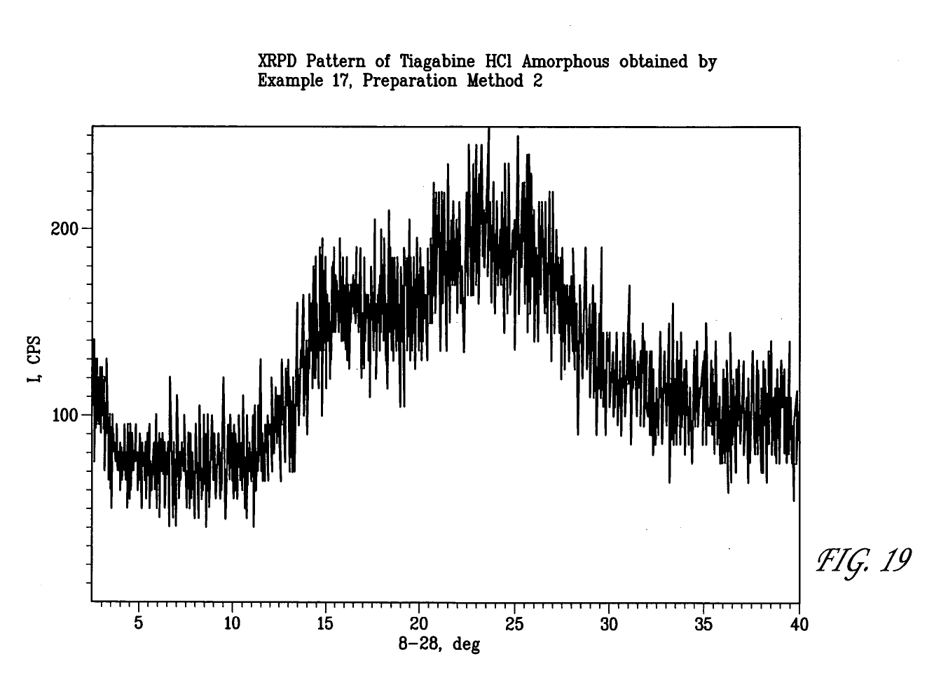

FIG. 19 depicts an XRPD pattern of tiagabine hydrochloride amorphous obtained by Example 17, Preparation Method 2.

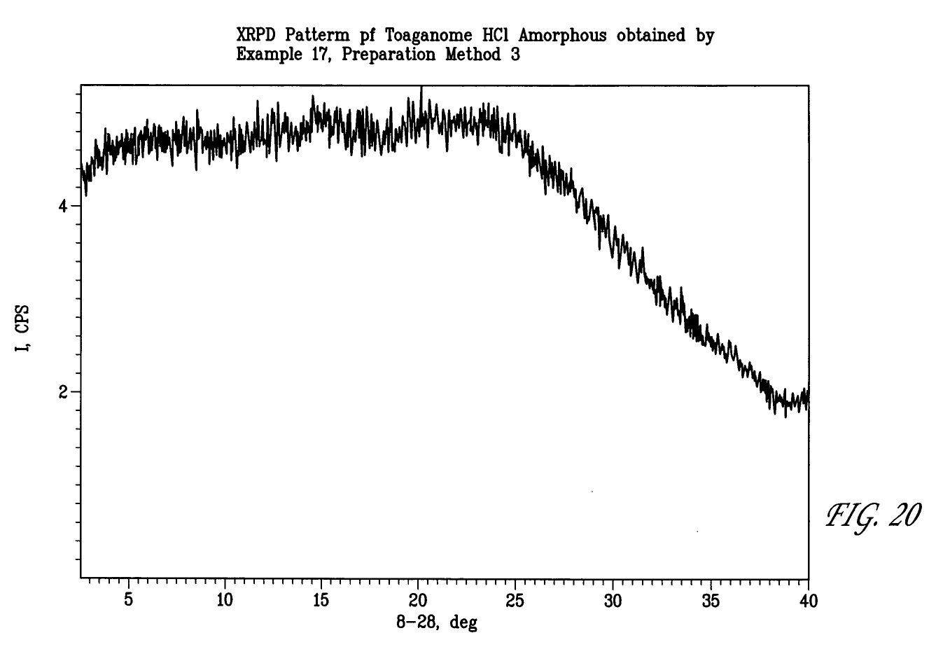

FIG. 20 depicts an XRPD pattern of tiagabine hydrochloride amorphous obtained by Example 17, Preparation Method 3.

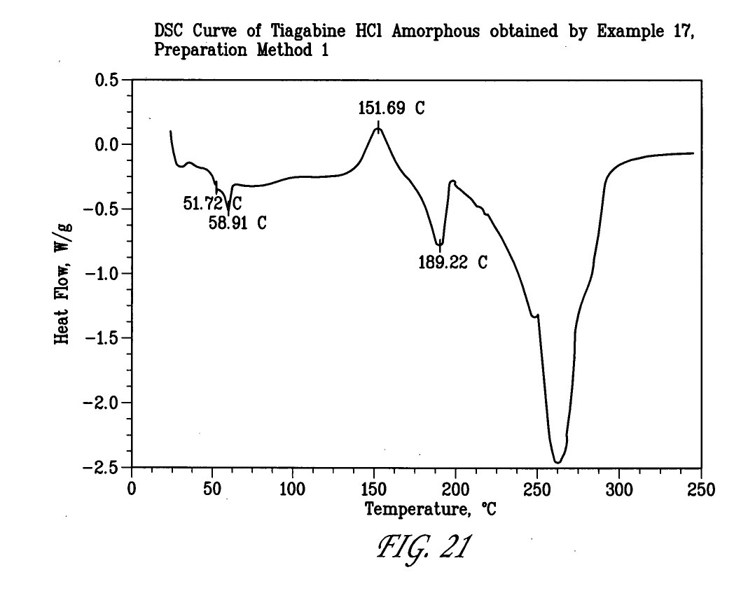

FIG. 21 depicts a DSC curve of tiagabine hydrochloride amorphous obtained by Example 17, Preparation Method 1.

### DETAILED DESCRIPTION OF THE INVENTION

Definitions

“Crystalline form” refers to a solid chemical compound that provides a pattern of peaks when analyzed by x-ray powder diffraction; this includes polymorphs, solvates, hydrates, and desolvated solvates; “purity” refers to the relative quantity by weight of one component in a mixture (% w/w); “solution” refers to a mixture containing at least one solvent and at least one compound at least partially dissolved in the solvent.

Preparation and Characterization

The present invention provides 16 new crystalline forms of tiagabine hydrochloride.

Tiagabine Hydrochloride Form C

Tiagabine hydrochloride Form C may be prepared by crystallizing tiagabine hydrochloride from isopropanol.

The XRPD pattern of tiagabine hydrochloride Form C contains peaks at 6.1, 7.9, 8.7, 12.7, 14.8, 16.1, 17.2, 22.9, 25.1, and 25.9±0.2 degrees 2θ. A representative XRPD pattern of tiagabine hydrochloride Form C is presented in FIG. 1.

Tiagabine hydrochloride Form C is stable for two (2) months when stored at ambient temperature and humidity.

Preferably, the tiagabine hydrochloride Form C of the present invention has a purity of at least about 50% (w/w). More preferably, the tiagabine hydrochloride Form C has a purity of at least about 70% (w/w). More preferably, the tiagabine hydrochloride Form C has a purity of at least about 90% (w/w).

Tiagabine Hydrochloride Form D

Tiagabine hydrochloride Form D may be prepared by crystallizing tiagabine hydrochloride from acetonitrile.

The XRPD pattern of tiagabine hydrochloride Form D contains peaks at 7.9, 12.7, 14.4, 16.9, 17.1, 18.1, 18.8, 21.5, 22.0, and 24.3±0.2 degrees 2θ. A representative XRPD pattern of tiagabine hydrochloride Form D is presented in FIG. 2. Tiagabine hydrochloride Form D is further characterized by a DSC curve having major endotherms at 117° C. and 195° C.

Tiagabine hydrochloride Form D converts to tiagabine hydrochloride Form B, sometimes mixed with tiagabine hydrochloride Form Q, during storage.

Preferably, the tiagabine hydrochloride Form D of the present invention has a purity of at least about 50% (w/w). More preferably, the tiagabine hydrochloride Form D has a purity of at least about 70% (w/w). More preferably, the tiagabine hydrochloride Form D has a purity of at least about 90% (w/w).

Tiagabine Hydrochloride Form H

Tiagabine hydrochloride Form H may be prepared by crystallizing tiagabine hydrochloride from methyl ethyl ketone.

The XRPD pattern of tiagabine hydrochloride Form H contains peaks at 5.8, 7.6, 7.8, 11.6, 14.6, 15.9, 17.0, 19.7, 22.6, and 25.1±0.2 degrees 2θ. A representative XRPD pattern of tiagabine hydrochloride Form H is presented in FIG. 3.

Preferably, the tiagabine hydrochloride Form H of the present invention has a purity of at least about 50% (w/w). More preferably, the tiagabine hydrochloride Form H has a purity of at least about 70% (w/w). More preferably, the tiagabine hydrochloride Form H has a purity of at least about 90% (w/w).

Tiagabine Hydrochloride Form I

Tiagabine hydrochloride Form I may be prepared by crystallizing tiagabine hydrochloride from acetone.

The XRPD pattern of tiagabine hydrochloride Form I contains peaks at 10.5, 12.5, 13.1, 15.0, 17.3, 20.6, 21.0, 24.8, 25.2, and 27.0±0.2 degrees 2θ. A representative XRPD pattern of tiagabine hydrochloride Form I is presented in FIG. 4.

Tiagabine hydrochloride Form I converts to a mixture of tiagabine hydrochloride Forms S and B during storage.

Preferably, the tiagabine hydrochloride Form I of the present invention has a purity of at least about 50% (w/w). More preferably, the tiagabine hydrochloride Form I has a purity of at least about 70% (w/w). More preferably, the tiagabine hydrochloride Form I has a purity of at least about 90% (w/w).

Tiagabine Hydrochloride Form J

Tiagabine hydrochloride Form J may be prepared by crystallizing tiagabine hydrochloride from ethanol.

The XRPD pattern of tiagabine hydrochloride Form J contains peaks at 7.8, 12.4, 13.0, 14.6, 17.0, 17.5, 21.1, 21.8, 24.8, and 26.2±0.2 degrees 2θ. A representative XRPD pattern of tiagabine hydrochloride Form J is presented in FIG. 5.

Tiagabine hydrochloride Form J converts to a mixture of tiagabine hydrochloride Forms Q and B during storage.

Preferably, the tiagabine hydrochloride Form J of the present invention has a purity of at least about 50% (w/w). More preferably, the tiagabine hydrochloride Form J has a purity of at least about 70% (w/w). More preferably, the tiagabine hydrochloride Form J has a purity of at least about 90% (w/w).

Tiagabine Hydrochloride Form M

Tiagabine hydrochloride Form M may be prepared by crystallizing tiagabine hydrochloride from dichloromethane.

The XRPD pattern of tiagabine hydrochloride Form M contains peaks at 7.8, 12.8, 14.5, 16.9, 21.1, 21.8, 24.5, 24.9, 26.3, and 27.5±0.2 degrees 2θ. A representative XRPD pattern of tiagabine hydrochloride Form M is presented in FIG. 6.

Tiagabine hydrochloride Form M converts to a mixture of tiagabine hydrochloride Forms B and Q during storage.

Preferably, the tiagabine hydrochloride Form M of the present invention has a purity of at least about 50% (w/w). More preferably, the tiagabine hydrochloride Form M has a purity of at least about 70% (w/w). More preferably, the tiagabine hydrochloride Form M has a purity of at least about 90% (w/w).

Tiagabine Hydrochloride Form P

Tiagabine hydrochloride Form P may be prepared by crystallizing tiagabine hydrochloride from 1,4-dioxane. Tiagabine hydrochloride Form P may be prepared by crystallizing tiagabine hydrochloride from methyl ethyl ketone.

The XRPD pattern of tiagabine hydrochloride Form P contains peaks at 12.5, 14.5, 16.1, 17.6, 21.9, 25.2, 26.5, 35.8, 37.7, and 39.3±0.2 degrees 2θ. A representative XRPD pattern of tiagabine hydrochloride Form P is presented in FIG. 7. Tiagabine hydrochloride Form P is further characterized by a DSC curve having a major endotherm at about 195° C.

Tiagabine hydrochloride Form P converts to Form B during storage.

Preferably, the tiagabine hydrochloride Form P of the present invention has a purity of at least about 50% (w/w). More preferably, the tiagabine hydrochloride Form P has a purity of at least about 70% (w/w). More preferably, the tiagabine hydrochloride Form P has a purity of at least about 90% (w/w).

Tiagabine Hydrochloride Form Q

Tiagabine hydrochloride Form Q may be prepared by crystallizing tiagabine hydrochloride from methyl t-butyl ether. Tiagabine hydrochloride Form Q may be prepared by crystallizing tiagabine hydrochloride from methanol. Tiagabine hydrochloride Form Q also may be prepared by drying tiagabine hydrochloride Form H in a vacuum oven.

The XRPD pattern of tiagabine hydrochloride Form Q contains peaks at 6.4, 11.4, 12.9, 14.8, 15.3, 16.7, 18.8, 22.9, 24.7, and 25.3±0.2 degrees 2θ. A representative XRPD pattern of tiagabine hydrochloride Form Q is presented in FIG. 8.

Preferably, the tiagabine hydrochloride Form Q of the present invention has a purity of at least about 50% (w/w). More preferably, the tiagabine hydrochloride Form Q has a purity of at least about 70% (w/w). More preferably, the tiagabine hydrochloride Form Q has a purity of at least about 90% (w/w).

Tiagabine Hydrochloride Form T

Tiagabine hydrochloride Form T may be prepared by crystallizing tiagabine hydrochloride from 2-butanol.

The XRPD pattern of tiagabine hydrochloride Form T contains peaks at 7.9, 8.6, 12.6, 15.9, 17.1, 18.3, 20.8, 22.2, 23.5, and 25.0±0.2 degrees 2θ. A representative XRPD pattern of tiagabine hydrochloride Form T is presented in FIG. 9.

Preferably, the tiagabine hydrochloride Form T of the present invention has a purity of at least about 50% (w/w). More preferably, the tiagabine hydrochloride Form T has a purity of at least about 70% (w/w). More preferably, the tiagabine hydrochloride Form T has a purity of at least about 90% (w/w).

Tiagabine Hydrochloride Form W

Tiagabine hydrochloride Form W may be prepared by crystallizing tiagabine hydrochloride from acetone, optionally in admixture with cyclohexane. In one embodiment, tiagabine hydrochloride Form W may be prepared by crystallizing tiagabine hydrochloride from a 1:1 (v/v) mixture of acetone and cyclohexane.

The XRPD pattern of tiagabine hydrochloride Form W contains peaks at 12.6, 13.2, 16.6, 17.0, 17.6, 18.6, 21.0, 23.9, 24.3, and 24.8±0.2 degrees 2θ. A representative XRPD pattern of tiagabine hydrochloride Form W is presented in FIG. 10.

Preferably, the tiagabine hydrochloride Form W of the present invention has a purity of at least about 50% (w/w). More preferably, the tiagabine hydrochloride Form W has a purity of at least about 70% (w/w). More preferably, the tiagabine hydrochloride Form W has a purity of at least about 90% (w/w).

Tiagabine Hydrochloride Form Y

Tiagabine hydrochloride Form Y may be prepared by crystallizing tiagabine hydrochloride from 1,4-dioxane.

The XRPD pattern of tiagabine hydrochloride Form Y contains peaks at 7.7, 11.6, 14.6, 16.7, 16.9, 18.6, 18.9, 21.4, 22.4, and 25.6±0.2 degrees 2θ. A representative XRPD pattern of tiagabine hydrochloride Form Y is presented in FIG. 11.

Preferably, the tiagabine hydrochloride Form Y of the present invention has a purity of at least about 50% (w/w). More preferably, the tiagabine hydrochloride Form Y has a purity of at least about 70% (w/w). More preferably, the tiagabine hydrochloride Form Y has a purity of at least about 90% (w/w).

Tiagabine Hydrochloride Form Z

Tiagabine hydrochloride Form Z may be prepared by crystallizing tiagabine hydrochloride from tetrahydrofuran.

The XRPD pattern of tiagabine hydrochloride Form Z contains peaks at 5.6, 8.3, 11.4, 11.7, 13.2, 16.4, 19.9, 20.7, and 23.9±0.2 degrees 2θ. A representative XRPD pattern of tiagabine hydrochloride Form Z is presented in FIG. 12.

Preferably, the tiagabine hydrochloride Form Z of the present invention has a purity of at least about 50% (w/w). More preferably, the tiagabine hydrochloride Form Z has a purity of at least about 70% (w/w). More preferably, the tiagabine hydrochloride Form Z has a purity of at least about 90% (w/w).

Tiagabine Hydrochloride Form AA

Tiagabine hydrochloride Form AA may be prepared by slurrying tiagabine hydrochloride monohydrate in acetone.

The XRPD pattern of tiagabine hydrochloride Form AA contains peaks at 7.4, 11.2, 13.1, 14.7, 16.6, 18.2, 20.0, 22.0, 22.4, and 24.0±0.2 degrees 2θ. A representative XRPD pattern of tiagabine hydrochloride Form AA is presented in FIG. 13.

Preferably, the tiagabine hydrochloride Form AA of the present invention has a purity of at least about 50% (w/w). More preferably, the tiagabine hydrochloride Form AA has a purity of at least about 70% (w/w). More preferably, the tiagabine hydrochloride Form AA has a purity of at least about 90% (w/w).

Tiagabine Hydrochloride Form S

Tiagabine hydrochloride Form S may be prepared by storing tiagabine hydrochloride Form I at room temperature for about two (2) months.

The XRPD pattern of tiagabine hydrochloride Form S contains peaks at 6.7, 7.9, 12.5, 13.1, 17.6, 21.8, and 27.7±0.2 degrees 2θ. A representative XRPD pattern of tiagabine hydrochloride Form S in admixture with tiagabine hydrochloride anhydrous is presented in FIG. 14.

Preferably, the tiagabine hydrochloride Form S of the present invention has a purity of at least about 50% (w/w). More preferably, the tiagabine hydrochloride Form S has a purity of at least about 70% (w/w). More preferably, the tiagabine hydrochloride Form S has a purity of at least about 90% (w/w).

Tiagabine Hydrochloride Form X

Tiagabine hydrochloride Form X may be prepared by crystallizing tiagabine hydrochloride from water.

The XRPD pattern of tiagabine hydrochloride Form X contains peaks at 7.8, 11.7, 14.0, 15.6, 18.5, 18.9, and 24.9±0.2 degrees 2θ. A representative XRPD pattern of tiagabine hydrochloride Form X in admixture with tiagabine hydrochloride Form A is presented in FIG. 16.

Preferably, the tiagabine hydrochloride Form X of the present invention has a purity of at least about 50% (w/w). More preferably, the tiagabine hydrochloride Form X has a purity of at least about 70% (w/w). More preferably, the tiagabine hydrochloride Form X has a purity of at least about 90% (w/w).

Tiagabine Hydrochloride Form AB

Tiagabine hydrochloride Form AB may be prepared by heating tiagabine hydrochloride monohydrate at 150° C.

The XRPD pattern of tiagabine hydrochloride Form AB contains peaks at 4.1, 7.6, 14.0, 17.8, and 18.4±0.2 degrees 2θ. A representative XRPD pattern of tiagabine hydrochloride Form AB in admixture with tiagabine hydrochloride anhydrous is presented in FIG. 17.

Preferably, the tiagabine hydrochloride Form AB of the present invention has a purity of at least about 50% (w/w). More preferably, the tiagabine hydrochloride Form AB has a purity of at least about 70% (w/w). More preferably, the tiagabine hydrochloride Form AB has a purity of at least about 90% (w/w).

Tiagabine Hydrochloride Amorphous

Tiagabine hydrochloride amorphous may be prepared by the steps of:
(a) heating tiagabine hydrochloride at or above its melting point, and
(b) cooling the heated tiagabine hydrochloride.
   Preferably, the cooling step (b) is performed by immersing a container of the melted tiagabine hydrochloride in an ice bath. Preferably, the cooling step (b) is performed by immersing a container of the melted tiagabine hydrochloride in a dry ice/isopropanol bath.

Tiagabine hydrochloride amorphous also may be prepared by the steps of:
(a) preparing an aqueous solution of tiagabine hydrochloride, and
(b) freeze drying the aqueous solution of tiagabine hydrochloride.

The XRPD pattern of tiagabine hydrochloride amorphous lacks individual peaks. Representative XRPD patterns of tiagabine hydrochloride amorphous are presented in FIGS. 18-20. Tiagabine hydrochloride amorphous is further characterized by a DSC curve having endotherms at 52° C., 59° C., and 189° C., and an exotherm at 152° C. A representative DSC curve of tiagabine hydrochloride amorphous is presented in FIG. 21.

Tiagabine hydrochloride amorphous is stable for at least 5 days and 8 days, respectively, when stored at about 5° C. and either 11% or 43% relative humidity. Tiagabine hydrochloride amorphous is stable for at least 22 days when stored at room temperature and either 33% or 58% relative humidity. Tiagabine hydrochloride amorphous converted to a mixture of Forms A and B when stored for 22 days at room temperature and either 75% or 84% relative humidity. Tiagabine hydrochloride amorphous converts to tiagabine hydrochloride anhydrous when heated at 160° C. in an argon atmosphere.

Preferably, the tiagabine hydrochloride amorphous of the present invention has a purity of at least about 50% (w/w). More preferably, the tiagabine hydrochloride amorphous has a purity of at least about 70% (w/w). More preferably, the tiagabine hydrochloride amorphous has a purity of at least about 90% (w/w).

Pharmaceutical Composition

The present invention provides a pharmaceutical composition comprising a pharmaceutically acceptable excipient and at least one tiagabine form chosen from tiagabine hydrochloride Forms C, D, H, I, J, M, P, Q, T, W, Y, Z, AA, S, X, and AB, and tiagabine hydrochloride amorphous form. Preferably, the tiagabine form is tiagabine hydrochloride Form C. Preferably, the tiagabine form is tiagabine hydrochloride Form D. Preferably, the tiagabine form is tiagabine hydrochloride Form H. Preferably, the tiagabine form is tiagabine hydrochloride Form I. Preferably, the tiagabine form is tiagabine hydrochloride Form J. Preferably, the tiagabine form is tiagabine hydrochloride Form M. Preferably, the tiagabine form is tiagabine hydrochloride Form P. Preferably, the tiagabine form is tiagabine hydrochloride Form Q. Preferably, the tiagabine form is tiagabine hydrochloride Form T. Preferably, the tiagabine form is tiagabine hydrochloride Form W. Preferably, the tiagabine form is tiagabine hydrochloride Form Y. Preferably, the tiagabine form is tiagabine hydrochloride Form Z. Preferably, the tiagabine form is tiagabine hydrochloride Form AA. Preferably, the tiagabine form is tiagabine hydrochloride Form S. Preferably, the tiagabine form is tiagabine hydrochloride Form X. Preferably, the tiagabine form is Form AB. Preferably, the tiagabine form is tiagabine hydrochloride amorphous form.

Further, there is provided a process for preparing such a pharmaceutical composition, comprising the step of mixing at least one tiagabine form chosen from tiagabine hydrochloride Forms C, D, H, I, J, M, P, Q, T, W, Y, Z, AA, S, X, and AB, and tiagabine hydrochloride amorphous form with a pharmaceutically acceptable excipient. Preferably, the tiagabine form is tiagabine hydrochloride Form C. Preferably, the tiagabine form is tiagabine hydrochloride Form D. Preferably, the tiagabine form is tiagabine hydrochloride Form H. Preferably, the tiagabine form is tiagabine hydrochloride Form I. Preferably, the tiagabine form is tiagabine hydrochloride Form J. Preferably, the tiagabine form is tiagabine hydrochloride Form M. Preferably, the tiagabine form is tiagabine hydrochloride Form P. Preferably, the tiagabine form is tiagabine hydrochloride Form Q. Preferably, the tiagabine form is tiagabine hydrochloride Form T. Preferably, the tiagabine form is tiagabine hydrochloride Form W. Preferably, the tiagabine form is tiagabine hydrochloride Form Y. Preferably, the tiagabine form is tiagabine hydrochloride Form Z. Preferably, the tiagabine form is tiagabine hydrochloride Form AA. Preferably, the tiagabine form is tiagabine hydrochloride Form S. Preferably, the tiagabine form is tiagabine hydrochloride Form X. Preferably, the tiagabine form is Form AB. Preferably, the tiagabine form is tiagabine hydrochloride amorphous form.

The present tiagabine forms may, for example, conveniently be formulated for topical, oral, buccal, sublingual, parenteral, local or rectal administration. Preferably, the pharmaceutical composition is a dry oral dosage form. Preferably, the pharmaceutical composition is an oral dosage form chosen from tablet, pill, capsule, caplet, powder, granule, and gel. Dry dosage forms may include pharmaceutically acceptable additives, such as excipients, carriers, diluents, stabilizers, plasticizers, binders, glidants, disintegrants, bulking agents, lubricants, plasticizers, colorants, film formers, flavoring agents, preservatives, dosing vehicles, and any combination of any of the foregoing.

Diluents increase the bulk of a solid pharmaceutical composition and may make a pharmaceutical dosage form containing the composition easier for the patient and caregiver to handle. Diluents for solid compositions include, but are not limited to, microcrystalline cellulose (e.g. AVICEL®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit), potassium chloride, powdered cellulose, sodium chloride, sorbitol and talc.

Binders for solid pharmaceutical compositions include, but are not limited to, acacia, alginic acid, carbomer (e.g. Carbopol), carboxymethylcellulose sodium, dextrin, ethyl cellulose, gelatin, guar gum, hydrogenated vegetable oil, hydroxyethyl cellulose, hydroxypropyl cellulose (e.g. KLUCEL®), hydroxypropyl methyl cellulose (e.g. METHOCEL®), liquid glucose, magnesium aluminum silicate, maltodextrin, methylcellulose, polymethacrylates, povidone (e.g. KOLLIDON®, PLASDONE®), pregelatinized starch, sodium alginate and starch.

The dissolution rate of a compacted solid pharmaceutical composition in the patient's stomach may be increased by the addition of a disintegrant to the composition. Disintegrants include, but are not limited to, alginic acid, carboxymethylcellulose calcium, carboxymethylcellulose sodium (e.g. AC-DI-SOL®, PRIMELLOSE®), colloidal silicon dioxide, croscarmellose sodium, crospovidone (e.g. KOLLIDON®, POLYPLASDONE®), guar gum, magnesium aluminum silicate, methyl cellulose, microcrystalline cellulose, powdered cellulose, pregelatinized starch, sodium alginate, sodium starch glycolate (e.g. EXPLOTAB®) and starch.

Glidants can be added to improve the flow properties of non-compacted solid compositions and improve the accuracy of dosing. Excipients that may function as glidants include, but are not limited to, colloidal silicon dioxide, magnesium trisilicate, powdered cellulose, starch, talc and tribasic calcium phosphate.

When a dosage form such as a tablet is made by compaction of a powdered composition, the composition is subjected to pressure from a punch and die. Some excipients and active ingredients have a tendency to adhere to the surfaces of the punch and die, which can cause the product to have pitting and other surface irregularities. A lubricant can be added to the composition to reduce adhesion and ease release of the product from the die. Lubricants include, but are not limited to, magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid ethyl maltol, and tartaric acid.

Compositions may also be colored using any pharmaceutically acceptable colorant to improve their appearance and/or facilitate patient identification of the product and unit dosage level.

Selection of excipients and the amounts to use may be readily determined by formulation scientists based upon experience and consideration of standard procedures and reference works in the field. The solid compositions of the present invention include powders, granulates, aggregates and compacted compositions. The preferred route of the present invention is oral. The dosages may be conveniently presented in unit dosage form and prepared by any of the methods well-known in the pharmaceutical arts. Dosage forms include solid dosage forms like tablets, pills, powders, caplets, granules, capsules, sachets, troches and lozenges. An especially preferred dosage form of the present invention is a tablet.

Ointments, creams and gels, may, for example, be formulated with an aqueous or oily base with the addition of a suitable thickening agent, gelling agent, and/or solvent. Such bases may thus, for example, include water and/or an oil such as liquid paraffin or a vegetable oil such as arachis oil or castor oil, or a solvent such as polyethylene glycol. Thickening agents and gelling agents that may be used according to the nature of the base include, but are not limited to, soft paraffin, aluminum stearate, cetostearyl alcohol, polyethylene glycols, woolfat, beeswax, carboxypolymethylene and cellulose derivatives, and/or glyceryl monostearate and/or non-ionic emulsifying agents.

Lotions may be formulated with an aqueous or oily base and will in general also contain one or more emulsifying agents, stabilizing agents, dispersing agents, suspending agents or thickening agents. Powders for external application may be formed with the aid of any suitable powder base, for example, talc, lactose or starch. Drops may be formulated with an aqueous or non-aqueous base also comprising one or more dispersing agents, solubilizing agents, suspending agents or preservatives.

If appropriate, the formulations of the invention may be buffered by the addition of suitable buffering agents.

Preferably, the pharmaceutical composition of the present invention is a unit dose composition. Preferably, the pharmaceutical composition of the present invention contains about 1 to 200 mg of the tiagabine form. More preferably, the pharmaceutical composition contains about 2 to 100 mg of the tiagabine form. More preferably, the pharmaceutical composition contains about 2 to 50 mg of the tiagabine form. More preferably, the pharmaceutical composition contains about 2 mg, 4 mg, 8 mg, 10 mg, 12 mg, 16 mg, 20 mg, 25 mg, or 30 mg of the tiagabine form. More preferably, the pharmaceutical composition contains about 2 mg, 4 mg, 12 mg, or 16 mg of the tiagabine form.

Method of Treatment

The present invention provides a method of treating a disease related to GABA uptake in a mammal, comprising the step of administering to the mammal a therapeutically effective amount of at least one tiagabine form chosen from tiagabine hydrochloride Forms C, D, H, I, J, M, P, Q, T, W, Y, Z, AA, S, X, and AB, and tiagabine hydrochloride amorphous form. Preferably, the tiagabine form is tiagabine hydrochloride Form C. Preferably, the tiagabine form is tiagabine hydrochloride Form D. Preferably, the tiagabine form is tiagabine hydrochloride Form H. Preferably, the tiagabine form is tiagabine hydrochloride Form I. Preferably, the tiagabine form is tiagabine hydrochloride Form J. Preferably, the tiagabine form is tiagabine hydrochloride Form M. Preferably, the tiagabine form is tiagabine hydrochloride Form P. Preferably, the tiagabine form is tiagabine hydrochloride Form Q. Preferably, the tiagabine form is tiagabine hydrochloride Form T. Preferably, the tiagabine form is tiagabine hydrochloride Form W. Preferably, the tiagabine form is tiagabine hydrochloride Form Y. Preferably, the tiagabine form is tiagabine hydrochloride Form Z. Preferably, the tiagabine form is tiagabine hydrochloride Form AA. Preferably, the tiagabine form is tiagabine hydrochloride Form S. Preferably, the tiagabine form is tiagabine hydrochloride Form X. Preferably, the tiagabine form is Form AB. Preferably, the tiagabine form is tiagabine hydrochloride amorphous form.

Preferably, the disease related to GABA uptake is at least one disease chosen from epilepsy and partial seizures. Preferably, the disease related to GABA uptake is epilepsy. Preferably, the disease related to GABA uptake is partial seizures.

Preferably, the therapeutically effective amount is 1 to 500 mg per day. More preferably, the therapeutically effective amount is 1 to 100 mg per day. More preferably, the therapeutically effective amount is 4 to 60 mg per day.

Methodology and Protocols

X-Ray Powder Diffraction

X-ray powder diffraction (XRPD) analyses were performed using the following instruments & methods:

A. Shimadzu XRD-6000 X-ray powder diffractometer using Cu Kα radiation. The instrument was equipped with a long fine focus X-ray tube. The tube voltage and amperage were set to 40 kV and 40 mA, respectively. The divergence and scattering slits were set at 1° and the receiving slit was set at 0.15 mm. Diffracted radiation was detected by a NaI scintillation detector. A θ-2θ continuous scan at 3°/min (0.4 sec/0.02° step) from 2.5 to 40° 2θ was used. A silicon standard was analyzed to check the instrument alignment. Data were collected and analyzed using XRD-6000 v. 4.1. Samples were prepared for analysis by placing them in a sample holder.

B. Inel XRG-3000 diffractometer, equipped with a CPS (Curved Position Sensitive) detector with a 2 Orange of 120°. Real time data were collected using Cu—Kα radiation starting at approximately 4° 2θ at a resolution of 0.03° 2θ. The tube voltage and amperage were set to 40 kV and 30 mA, respectively. The monochromator slit was set at 5 mm by 80 μm or 160 μm. The pattern is displayed from 2.5-40° 2θ. Samples were prepared for analysis by packing them into thin-walled glass capillaries. Each capillary was mounted onto a goniometer head that is motorized to permit spinning of the capillary during data acquisition. The acquisition time was between 5 to 10 min. Instrument calibration was performed using a silicon reference standard.

C. Shimadzu XRD-6000 X-ray powder diffractometer equipped with an Anton Paar HTK 1200 high temperature stage (Variable-temperature XRPD (VT-XRPD)). The sample was packed in a ceramic holder and analyzed form 2.5 to 40° 2θ at 3°/min (0.4 sec/0.02° step). The heating rate was 10° C./min. A silicon standard was analyzed to check the instrument alignment. Temperature calibration was performed using vanillin and sulfapyridine USP melting point standards. Data were collected and analyzed using XPD-6000 v.4.1. The system was kept under a purge of nitrogen during the analysis.

D. Bruker D-8 Discover diffractometer and Bruker's General Area Diffraction Detection System (GADDS, v. 4.1.20). An incident beam of Cu—Kα radiation was produced using a fine-focus tube (40 kV, 40 mA), a Gobel mirror, and a 0.5 mm double-pinhole collimator. The samples were positioned for analysis by securing the well plate to a translation stage and moving each sample to intersect the incident beam. Alternatively, the sample was packed between 3-micron thick films to form a portable disc-shaped specimen, and the specimen was loaded in a holder secured to a translation stage. The samples were analyzed using a transmission geometry. The incident beam was scanned and rastered over the sample during the analysis to optimize orientation statistics. A beam-stop was used to minimize air scatter from the incident beam at low angles. Diffraction patterns were collected using a Hi-Star area detector located 15 cm from the sample and processed using GADDS. The intensity in the GADDS image of the diffraction pattern was integrated using a step size of 0.04° 2θ. The integrated patterns display diffraction intensity as a function of 2θ. Prior to the analysis a silicon standard was analyzed to verify the Si 111 peak position.

E. Peak Picking Methods. Any XRPD files generated from Inel or Bruker XRPD instruments were converted to Shimadzu .raw file using File Monkey version 3.0.4. The Shimadzu .raw file was processed by the Shimadzu XRD-6000 version 4.1 software to automatically find peak positions. The “peak position” means the maximum intensity of a peaked intensity profile. The following processes were used with the Shimadzu XRD-6000 “Basic Process” version 2.6 algorithm:
Smoothing was done on all patterns.
The background was subtracted to find the net, relative intensity of the peaks.
A peak from Cu K alpha2 (1.5444 Å) wavelength was subtracted from the peak generated by Cu K alpha1 (1.5406 Å) peak at 50% intensity for all patterns.
   Differential Scanning Calorimetry

Differential scanning calorimetry (DSC) was performed using a TA Instruments differential scanning calorimeter 2920. The sample was placed into an aluminum DSC pan, and the weight accurately recorded. The pan was covered with a lid and then crimped. The sample cell was equilibrated at ambient temperature and heated under a nitrogen purge at a rate of 10° C./min, up to a final temperature of 350° C. Indium metal was used as the calibration standard. Reported temperatures are at the transition maxima.

Thermogravimetry

Standard thermogravimetry (TG) analyses were performed using a TA Instruments 2950 thermogravimetric analyzer. Each sample was placed in an aluminum sample pan and inserted into the TG furnace. The furnace was optionally equilibrated at 25° C. then heated under nitrogen at a rate of 10° C./min, up to a final temperature of 300° C. or 350° C. Nickel and Alumel™ were used as the calibration standards.

Proton Solution Nuclear Magnetic Resonance

The solution ¹H nuclear magnetic resonance (NMR) spectrum was acquired at ambient temperature with a Varian ^{UNITY}INOVA-400 spectrometer at a ¹H Larmor frequency of 399.80 MHz. The sample was dissolved in DMSO-d₆ or CDCl₃. The free induction decay (FID) was processed using the Varian VNMR 6.1B software with 3200 to 131072 points and an exponential line broadening factor of 0.20 Hz to improve the signal-to-noise ratio. The spectrum was referenced to internal tetramethylsilane (TMS).

Moisture Sorption/Desorption

Moisture sorption/desorption data were collected on a VTI SGA-100 moisture balance system. For sorption isotherms, a sorption range of 5 to 95% relative humidity (RH) and a desorption range of 95 to 5% RH in 10% RH increments were used for analysis. The samples were not dried prior to analysis. Equilibrium criteria used for analysis were less than 0.0100% weight change in 5 minutes with a maximum equilibration time of 3 hours if the weight criterion was not met. Data were not corrected for the initial moisture content of the samples.

### EXAMPLES

### Example 1

### Preparation and Characterization of Tiagabine Hydrochloride Form C

Preparation Method 1

Approximately 127 mg of tiagabine HCl monohydrate was dissolved in approximately 0.75 mL of 2-propanol. A clear solution was obtained at first and solid quickly precipitated out. The sample was capped and placed in hood at ambient temperature overnight. The liquid was decanted and the remaining solids were air dried.

Preparation Method 2

A saturated solution of tiagabine HCl monohydrate in 2-propanol was filtered through a 0.2 μm nylon filter into a vial. The resulting solution in an open vial was allowed to evaporate quickly until dryness. A white, needle-like, solid was obtained.

XRPD

A representative XRPD pattern of tiagabine hydrochloride Form C is presented in FIG. 1. Representative peaks are listed in the following Table 2.

TABLE 2
Tiagabine HCl Form C XRPD Peaks

| Peak No.^{a} | Position (°2θ) | d-spacing | Intensity | I/I₀^{b} |
|---|---|---|---|---|
| 1 | 6.1 | 14.6 | 243 | 23 |
| 2 | 7.9 | 11.2 | 278 | 26 |
| 3 | 8.7 | 10.2 | 380 | 35 |
| 4 | 11.1 | 8.0 | 102 | 9 |
| 5 | 12.7 | 6.9 | 479 | 44 |
| 6 | 13.1 | 6.7 | 67 | 6 |
| 7 | 13.9 | 6.4 | 119 | 11 |
| 8 | 14.8 | 6.0 | 225 | 21 |
| 9 | 15.2 | 5.8 | 128 | 12 |
| 10 | 16.1 | 5.5 | 365 | 34 |
| 11 | 16.8 | 5.3 | 101 | 9 |
| 12 | 17.2 | 5.2 | 1080 | 100 |
| 13 | 17.8 | 5.0 | 157 | 15 |
| 14 | 18.3 | 4.9 | 109 | 10 |
| 15 | 19.3 | 4.6 | 178 | 16 |
| 16 | 19.9 | 4.5 | 78 | 7 |
| 17 | 20.1 | 4.4 | 82 | 8 |
| 18 | 20.9 | 4.2 | 105 | 10 |
| 19 | 21.7 | 4.1 | 165 | 15 |
| 20 | 22.2 | 4.0 | 102 | 9 |
| 21 | 22.9 | 3.9 | 300 | 28 |
| 22 | 23.4 | 3.8 | 197 | 18 |
| 23 | 23.7 | 3.7 | 168 | 16 |
| 24 | 24.0 | 3.7 | 93 | 9 |
| 25 | 25.1 | 3.5 | 553 | 51 |
| 26 | 25.4 | 3.5 | 263 | 24 |
| 27 | 25.9 | 3.4 | 321 | 30 |
| 28 | 26.3 | 3.4 | 151 | 14 |
| 29 | 27.9 | 3.2 | 117 | 11 |
| 30 | 28.2 | 3.2 | 116 | 11 |
| 31 | 30.8 | 2.9 | 69 | 6 |

^{a}Bold: Unique set of XRPD Peaks for Form C.
^{b}Intensity of peak/Intensity of most intense peak

Stability

Tiagabine HCl Form C was stored for two months under conditions of ambient temperature and humidity. XRPD analysis of the resulting sample indicated tiagabine HCl Form C.

### Example 2

### Preparation and Characterization of Tiagabine Hydrochloride Form D

Preparation Method 1

A mixture of 99 mg of tiagabine HCl monohydrate and 10 mL of acetonitrile was heated at reflux on a hotplate for about 30 min to give a clear solution. The resulting solution was left on the hotplate and allowed to slow cool to ambient after the heating was discontinued. The liquid was decanted and the remaining white solids were air dried.

Preparation Method 2

A saturated solution of tiagabine HCl monohydrate in acetonitrile was filtered through a 0.2 μm nylon filter into a vial. The resulting solution in an open vial was allowed to evaporate quickly until dryness. A white, blade-like, solid was obtained.

Preparation Method 3

A mixture of 122 mg of tiagabine HCl monohydrate and 4 mL of acetonitrile was slurried for 4 days at room temperature. A white solid was collected by filtration and air dried.

Preparation Method 4

Tiagabine HCl monohydrate (88 mg) was dissolved in acetonitrile/water (1/1, v/v) and filtered through a 0.2 μm filter. The solvent was allowed to evaporate under ambient conditions. The resulting gummy residue was treated with acetonitrile (1 mL) and the sample placed on a shaker block. Solids formed after approximately two (2) hours and were collected by decantation of the liquid phase after one (1) day.

XRPD

A representative XRPD pattern of tiagabine hydrochloride Form D is presented in FIG. 2. Representative peaks are listed in the following Table 3.

TABLE 3
Tiagabine HCl Form D XRPD Peaks

| Peak No.^{a} | Position (°2θ) | d-spacing | Intensity | I/I₀^{b} |
|---|---|---|---|---|
| 1 | 7.6 | 11.6 | 99 | 9 |
| 2 | 7.9 | 11.2 | 308 | 27 |
| 3 | 12.7 | 7.0 | 1148 | 100 |
| 4 | 13.4 | 6.6 | 130 | 11 |
| 5 | 14.4 | 6.2 | 837 | 73 |
| 6 | 15.6 | 5.7 | 190 | 17 |
| 7 | 15.7 | 5.6 | 120 | 10 |
| 8 | 16.9 | 5.3 | 484 | 42 |
| 9 | 17.1 | 5.2 | 530 | 46 |
| 10 | 17.5 | 5.1 | 182 | 16 |
| 11 | 18.1 | 4.9 | 392 | 34 |
| 12 | 18.3 | 4.8 | 75 | 7 |
| 13 | 18.8 | 4.7 | 483 | 42 |
| 14 | 19.7 | 4.5 | 82 | 7 |
| 15 | 19.9 | 4.5 | 109 | 9 |
| 16 | 21.5 | 4.1 | 455 | 40 |
| 17 | 22.0 | 4.0 | 620 | 54 |
| 18 | 23.3 | 3.8 | 125 | 11 |
| 19 | 24.3 | 3.7 | 835 | 73 |
| 20 | 24.6 | 3.6 | 477 | 42 |
| 21 | 24.9 | 3.6 | 437 | 38 |
| 22 | 25.6 | 3.5 | 353 | 31 |
| 23 | 26.2 | 3.4 | 261 | 23 |
| 24 | 26.7 | 3.3 | 295 | 26 |
| 25 | 27.2 | 3.3 | 204 | 18 |
| 26 | 27.5 | 3.2 | 351 | 31 |
| 27 | 27.8 | 3.2 | 719 | 63 |
| 28 | 28.4 | 3.1 | 85 | 7 |
| 29 | 28.8 | 3.1 | 55 | 5 |
| 30 | 29.0 | 3.1 | 83 | 7 |
| 31 | 29.7 | 3.0 | 97 | 8 |
| 32 | 30.2 | 3.0 | 64 | 6 |
| 33 | 30.6 | 2.9 | 153 | 13 |
| 34 | 31.5 | 2.8 | 61 | 5 |
| 35 | 32.7 | 2.7 | 84 | 7 |
| 36 | 36.0 | 2.5 | 63 | 5 |
| 37 | 36.4 | 2.5 | 74 | 6 |
| 38 | 37.4 | 2.4 | 61 | 5 |
| 39 | 38.1 | 2.4 | 94 | 8 |
| 40 | 38.7 | 2.3 | 91 | 8 |

^{a}Bold: Unique set of XRPD Peaks for Form D.
^{b}Intensity of peak/Intensity of most intense peak × 100

DSC

DSC analysis of tiagabine HCl Form D indicated endotherms at 99° C. (broad, minor), 117° C. (broad, minor), 144° C. (minor, sharp), and 195° C. (major, sharp).

Stability

Tiagabine HCl Form D was stored for two months under conditions of ambient temperature and humidity. XRPD analysis of the resulting sample indicated a mixture of tiagabine HCl Forms Q and B.

A sample of tiagabine HCl Form D containing a minor amount of Form B was stored for five (5) days at about 60° C. and about 75% relative humidity. XRPD analysis of the resulting brownish sample indicated tiagabine HCl Form B and a trace of Q.

A sample of tiagabine HCl Form D containing a minor amount of Form B was stored for five (5) days at about 40° C. and about 89% relative humidity. XRPD analysis of the resulting brownish sample indicated a mixture of tiagabine HCl Form B and a trace of Q.

A sample of tiagabine HCl Form D was stored for five (5) days at 2-8° C. and about 96% relative humidity. XRPD analysis of the resulting white small needles indicated a mixture of tiagabine HCl Forms B and Q.

A sample of tiagabine HCl Form D was dried under vacuum at room temperature for less than one day. XRPD analysis of the resulting sample indicated a mixture of Form D containing a minor amount of Form A.

### Example 3

### Preparation and Characterization of Tiagabine Hydrochloride Form H

Approximately 27 mg of tiagabine HCl amorphous was dissolved in approximately 0.05 mL of methyl ethyl ketone. A clear solution was obtained at first and solids quickly precipitated out. The solvent was dried off by a stream of nitrogen and a white solid was obtained.

XRPD

A representative XRPD pattern of tiagabine hydrochloride Form H is presented in FIG. 3. Representative peaks are listed in the following Table 4.

TABLE 4
Tiagabine HCl Form H XRPD Peaks

| Peak No.^{a} | Position (°2θ) | d-spacing | Intensity | I/I₀^{b} |
|---|---|---|---|---|
| 1 | 5.8 | 15.3 | 179 | 16 |
| 2 | 7.6 | 11.6 | 455 | 41 |
| 3 | 7.8 | 11.4 | 496 | 44 |
| 4 | 11.1 | 8.0 | 92 | 8 |
| 5 | 11.4 | 7.7 | 99 | 9 |
| 6 | 11.6 | 7.6 | 340 | 30 |
| 7 | 13.8 | 6.4 | 163 | 15 |
| 8 | 14.6 | 6.1 | 1119 | 100 |
| 9 | 14.9 | 5.9 | 91 | 8 |
| 10 | 15.3 | 5.8 | 125 | 11 |
| 11 | 15.5 | 5.7 | 265 | 24 |
| 12 | 15.9 | 5.6 | 387 | 35 |
| 13 | 16.7 | 5.3 | 543 | 49 |
| 14 | 17.0 | 5.2 | 620 | 55 |
| 15 | 18.7 | 4.7 | 204 | 18 |
| 16 | 18.9 | 4.7 | 257 | 23 |
| 17 | 19.7 | 4.5 | 373 | 33 |
| 18 | 20.0 | 4.4 | 102 | 9 |
| 19 | 20.7 | 4.3 | 79 | 7 |
| 20 | 21.4 | 4.1 | 285 | 25 |
| 21 | 22.0 | 4.0 | 310 | 28 |
| 22 | 22.6 | 3.9 | 521 | 47 |
| 23 | 23.0 | 3.9 | 98 | 9 |
| 24 | 24.7 | 3.6 | 86 | 8 |
| 25 | 25.1 | 3.5 | 602 | 54 |
| 26 | 25.5 | 3.5 | 288 | 26 |
| 27 | 25.8 | 3.5 | 395 | 35 |
| 28 | 26.1 | 3.4 | 188 | 17 |
| 29 | 26.3 | 3.4 | 278 | 25 |
| 30 | 26.6 | 3.3 | 102 | 9 |
| 31 | 27.5 | 3.2 | 296 | 26 |
| 32 | 27.7 | 3.2 | 90 | 8 |
| 33 | 28.5 | 3.1 | 93 | 8 |
| 34 | 30.2 | 3.0 | 94 | 8 |
| 35 | 30.4 | 2.9 | 80 | 7 |
| 36 | 31.7 | 2.8 | 73 | 7 |
| 37 | 33.2 | 2.7 | 88 | 8 |
| 38 | 35.3 | 2.5 | 85 | 8 |
| 39 | 37.7 | 2.4 | 75 | 7 |
| 40 | 38.9 | 2.3 | 82 | 7 |

^{a}Bold: Unique set of XRPD Peaks for Form H.
^{b}Intensity of peak/Intensity of most intense peak × 100

TGA

TGA analysis indicated a 1.8% weight loss between 25 to 150° C.

Stability

Form H was stored at room temperature under vacuum for four (4) days. XRPD analysis of the resulting sample indicated Form Q.

Form H was heated at 90-95° C. for 10 minutes. XRPD analysis of the resulting sample indicated Form Q containing a minor amount of Form B.

### Example 4

### Preparation and Characterization of Tiagabine Hydrochloride Form I

A mixture of 103 mg of tiagabine HCl monohydrate and 10 mL of acetone was heated at reflux on a hotplate to give a clear solution. The resulting solution was left on the hotplate and allowed to slow cool to ambient temperature after the heating was discontinued. The liquid was decanted and the remaining white solids were air dried.

XRPD

A representative XRPD pattern of tiagabine hydrochloride Form I is presented in FIG. 4. Representative peaks are listed in the following Table 5.

TABLE 5
Tiagabine HCl Form I XRPD Peaks

| Peak No.^{a} | Position (°2θ) | d-spacing | Intensity | I/I₀^{b} |
|---|---|---|---|---|
| 1 | 4.2 | 21.0 | 114 | 23 |
| 2 | 10.5 | 8.4 | 234 | 47 |
| 3 | 11.1 | 7.9 | 125 | 25 |
| 4 | 12.1 | 7.3 | 204 | 41 |
| 5 | 12.5 | 7.0 | 438 | 88 |
| 6 | 13.1 | 6.8 | 500 | 100 |
| 7 | 14.8 | 6.0 | 175 | 35 |
| 8 | 15.0 | 5.9 | 372 | 74 |
| 9 | 15.5 | 5.7 | 53 | 11 |
| 10 | 15.9 | 5.6 | 72 | 14 |
| 11 | 16.6 | 5.3 | 186 | 37 |
| 12 | 17.3 | 5.1 | 336 | 67 |
| 13 | 17.7 | 5.0 | 96 | 19 |
| 14 | 18.2 | 4.9 | 93 | 19 |
| 15 | 18.6 | 4.8 | 78 | 16 |
| 16 | 19.3 | 4.6 | 132 | 26 |
| 17 | 20.0 | 4.4 | 178 | 36 |
| 18 | 20.6 | 4.3 | 309 | 62 |
| 19 | 21.0 | 4.2 | 370 | 74 |
| 20 | 21.5 | 4.1 | 104 | 21 |
| 21 | 21.9 | 4.1 | 61 | 12 |
| 22 | 22.6 | 3.9 | 65 | 13 |
| 23 | 23.6 | 3.8 | 209 | 42 |
| 24 | 24.4 | 3.6 | 211 | 42 |
| 25 | 24.8 | 3.6 | 305 | 61 |
| 26 | 25.2 | 3.5 | 307 | 61 |
| 27 | 25.7 | 3.5 | 173 | 35 |
| 28 | 26.4 | 3.4 | 110 | 22 |
| 29 | 27.0 | 3.3 | 323 | 65 |
| 30 | 27.3 | 3.3 | 114 | 23 |
| 31 | 27.8 | 3.2 | 81 | 16 |
| 32 | 28.3 | 3.2 | 79 | 16 |
| 33 | 29.6 | 3.0 | 111 | 22 |
| 34 | 30.5 | 2.9 | 70 | 14 |
| 35 | 30.9 | 2.9 | 80 | 16 |
| 36 | 32.4 | 2.8 | 55 | 11 |
| 37 | 32.7 | 2.7 | 90 | 18 |

^{a}Bold: Unique set of XRPD Peaks for Form I.
^{b}Intensity of peak/Intensity of most intense peak × 100

Stability

Tiagabine HCl Form I was stored for two months under conditions of ambient temperature and humidity. XRPD analysis of the resulting sample indicated a mixture of tiagabine HCl Forms S and B.

### Example 5

### Preparation and Characterization of Tiagabine Hydrochloride Form J

Preparation Method 1

Approximately 98 mg of tiagabine HCl monohydrate was dissolved in approximately 1 mL of EtOH to give a clear solution. The solution was placed in a refrigerator overnight. The liquid was decanted and the remaining solids were air dried.

Preparation Method 2

A mixture of 180 mg of tiagabine HCl monohydrate and 3 mL of EtOH was slurried for 4 days at room temperature. The white solids were collected by filtration and air dried.

XRPD

A representative XRPD pattern of tiagabine hydrochloride Form J is presented in FIG. 5. Representative peaks are listed in the following Table 6.

TABLE 6
Tiagabine HCl Form J XRPD Peaks

| Peak No.^{a} | Position (°2θ) | d-spacing | Intensity | I/I₀^{b} |
|---|---|---|---|---|
| 1 | 7.8 | 11.4 | 625 | 38 |
| 2 | 12.4 | 7.2 | 1642 | 100 |
| 3 | 13.0 | 6.8 | 554 | 34 |
| 4 | 13.6 | 6.5 | 97 | 6 |
| 5 | 14.1 | 6.3 | 167 | 10 |
| 6 | 14.6 | 6.1 | 863 | 53 |
| 7 | 14.9 | 5.9 | 77 | 5 |
| 8 | 15.5 | 5.7 | 194 | 12 |
| 9 | 16.5 | 5.4 | 204 | 12 |
| 10 | 17.0 | 5.2 | 1403 | 85 |
| 11 | 17.5 | 5.1 | 417 | 25 |
| 12 | 18.0 | 4.9 | 331 | 20 |
| 13 | 18.7 | 4.7 | 227 | 14 |
| 14 | 19.4 | 4.6 | 139 | 8 |
| 15 | 20.7 | 4.3 | 75 | 5 |
| 16 | 21.1 | 4.2 | 780 | 48 |
| 17 | 21.8 | 4.1 | 694 | 42 |
| 18 | 23.1 | 3.8 | 330 | 20 |
| 19 | 23.6 | 3.8 | 229 | 14 |
| 20 | 23.9 | 3.7 | 308 | 19 |
| 21 | 24.3 | 3.7 | 237 | 14 |
| 22 | 24.5 | 3.6 | 240 | 15 |
| 23 | 24.8 | 3.6 | 1037 | 63 |
| 24 | 25.1 | 3.5 | 300 | 18 |
| 25 | 26.0 | 3.4 | 224 | 14 |
| 26 | 26.2 | 3.4 | 486 | 30 |
| 27 | 26.7 | 3.3 | 226 | 14 |
| 28 | 27.3 | 3.3 | 305 | 19 |
| 29 | 27.5 | 3.2 | 316 | 19 |
| 30 | 27.8 | 3.2 | 394 | 24 |
| 31 | 28.2 | 3.2 | 136 | 8 |
| 32 | 28.4 | 3.1 | 117 | 7 |
| 33 | 28.6 | 3.1 | 143 | 9 |
| 34 | 29.2 | 3.1 | 177 | 11 |
| 35 | 29.4 | 3.0 | 101 | 6 |
| 36 | 30.0 | 3.0 | 171 | 10 |
| 37 | 30.8 | 2.9 | 201 | 12 |
| 38 | 35.9 | 2.5 | 117 | 7 |
| 39 | 37.6 | 2.4 | 136 | 8 |

^{a}Bold: Unique set of XRPD Peaks for Form J.
^{b}Intensity of peak/Intensity of most intense peak × 100

TGA

TGA analysis indicated a 7.0% weight loss between 25 to 150° C.

Stability

Tiagabine HCl Form J was stored for two months under conditions of ambient temperature and humidity. XRPD analysis of the resulting sample indicated a mixture of tiagabine HCl Forms Q and B.

### Example 6

### Preparation and Characterization of Tiagabine Hydrochloride Form M

Preparation Method 1

A mixture of 120 mg of tiagabine HCl monohydrate and 5 mL of dichloromethane was slurried for 1 day at room temperature. The white solids were collected by filtration and air dried.

Preparation Method 2

Amorphous tiagabine HCl (37.3 mg) was treated with dichloromethane (1,100 μL). The resulting waxy gel was slurried at ambient temperature for one day. Solvent was removed by decantation and solids dried under a gentle nitrogen stream.

Preparation Method 3

A mixture of tiagabine HCl monohydrate (88 mg) and dichloromethane (4 mL) was slurried for four (4) days at room temperature. The white solids were collected by filtration and air dried.

XRPD

A representative XRPD pattern of tiagabine hydrochloride Form M is presented in FIG. 6. Representative peaks are listed in the following Table 7.

TABLE 7
Tiagabine HCl Form M XRPD Peaks

| Peak No.^{a} | Position (°2θ) | d-spacing | Intensity | I/I₀^{b} |
|---|---|---|---|---|
| 1 | 7.8 | 11.4 | 164 | 12 |
| 2 | 12.4 | 7.1 | 219 | 15 |
| 3 | 12.8 | 6.9 | 387 | 27 |
| 4 | 13.1 | 6.7 | 133 | 9 |
| 5 | 14.5 | 6.1 | 790 | 56 |
| 6 | 15.6 | 5.7 | 85 | 6 |
| 7 | 16.6 | 5.3 | 341 | 24 |
| 8 | 16.9 | 5.2 | 1413 | 100 |
| 9 | 17.4 | 5.1 | 240 | 17 |
| 10 | 17.9 | 4.9 | 178 | 13 |
| 11 | 18.7 | 4.7 | 278 | 20 |
| 12 | 19.5 | 4.5 | 250 | 18 |
| 13 | 20.6 | 4.3 | 88 | 6 |
| 14 | 21.1 | 4.2 | 555 | 39 |
| 15 | 21.8 | 4.1 | 682 | 48 |
| 16 | 23.1 | 3.8 | 272 | 19 |
| 17 | 23.7 | 3.7 | 148 | 10 |
| 18 | 24.0 | 3.7 | 168 | 12 |
| 19 | 24.5 | 3.6 | 514 | 36 |
| 20 | 24.9 | 3.6 | 677 | 48 |
| 21 | 25.8 | 3.4 | 238 | 17 |
| 22 | 26.3 | 3.4 | 482 | 34 |
| 23 | 26.8 | 3.3 | 131 | 9 |
| 24 | 27.0 | 3.3 | 94 | 7 |
| 25 | 27.5 | 3.2 | 507 | 36 |
| 26 | 27.9 | 3.2 | 336 | 24 |
| 27 | 28.5 | 3.1 | 194 | 14 |
| 28 | 29.1 | 3.1 | 180 | 13 |
| 29 | 30.1 | 3.0 | 97 | 7 |
| 30 | 30.6 | 2.9 | 121 | 9 |
| 31 | 37.6 | 2.4 | 121 | 9 |

^{a}Bold: Unique set of XRPD Peaks for Form M.
^{b}Intensity of peak/Intensity of most intense peak × 100

TGA

TGA analysis indicated a two step weight loss of 1.6% between 25-50° C. and 8.7% weight loss between 50-150° C.

Stability

Tiagabine HCl Form M was stored for two months under conditions of ambient temperature and humidity. XRPD analysis of the resulting sample indicated a mixture of tiagabine HCl Forms B and Q.

### Example 7

### Preparation and Characterization of Tiagabine Hydrochloride Form P

Preparation Method 1

A mixture of 99 mg of tiagabine HCl monohydrate and 5 mL of 1,4-dioxane was slurried for 6 days at room temperature. The white solids were collected by filtration and air dried.

Preparation Method 2

Approximately 110 mg of tiagabine HCl monohydrate was dissolved in a mixture of 2 mL of 1,4-dioxane and 0.1 mL of water to provide a clear solution. The resulting solution in an open vial was allowed to evaporate quickly until dryness. A white, short needle, solid was obtained. XRPD analysis indicated a mixture of Form P with a minor amount of Form B.

Preparation Method 3

A mixture of 121 mg of tiagabine HCl monohydrate and 3 mL of methyl ethyl ketone was heated at reflux on a hotplate for about 10 min to give a clear solution. The resulting solution was left on the hotplate and allowed to cool slowly to ambient temperature after the heating was discontinued. The resulting clear solution was then placed in a refrigerator. The liquid was decanted and the remaining off-white solid was air dried. XRPD analysis indicated a mixture of Form P with a minor amount of Form B.

Preparation Method 4

Tetrahydrofuran (2.0 mL) was added to tiagabine HCl monohydrate (62 mg). The solids dissolved and then recrystallized to give a thick suspension. Water (100 μL) was added and the mixture was shaken and sonicated to give a clear solution. The vial was left uncapped and the solvent allowed to evaporate under ambient conditions for three (3) days, giving a gummy residue. Tetrahydrofuran (1 mL) was added, the vial capped and placed on a shaker block (ambient temperature). Solids formed after approximately two (2) hours and the slurry remained on the shaker block at ambient temperature for one day. The solids were collected by decantation of the solvent and air dried for approximately one (1) day. XRPD analysis indicated a mixture of Form P and Form B.

Preparation Method 5

Amorphous tiagabine HCl (9.7 mg) was dissolved in a mixture of 1,4-dioxane (20 μL) and water (7 μL). Solids formed over four (4) days at which time the vial was uncapped and the solvent allowed to evaporate.

XRPD

An XRPD pattern of tiagabine hydrochloride Form P obtained by Preparation Method 1 is presented in FIG. 7. Representative peaks are listed in the following Table 8.

TABLE 8
Tiagabine HCl Form P XRPD Peaks

| Peak No.^{a} | Position (°2θ) | d-spacing | Intensity | I/I₀^{b} |
|---|---|---|---|---|
| 1 | 7.9 | 11.1 | 33 | 10 |
| 2 | 12.5 | 7.1 | 167 | 50 |
| 3 | 13.2 | 6.7 | 68 | 21 |
| 4 | 14.5 | 6.1 | 273 | 82 |
| 5 | 15.5 | 5.7 | 73 | 22 |
| 6 | 16.1 | 5.5 | 331 | 100 |
| 7 | 17.3 | 5.1 | 92 | 28 |
| 8 | 17.6 | 5.0 | 209 | 63 |
| 9 | 18.0 | 4.9 | 151 | 46 |
| 10 | 18.3 | 4.8 | 76 | 23 |
| 11 | 18.7 | 4.7 | 51 | 15 |
| 12 | 19.4 | 4.6 | 106 | 32 |
| 13 | 19.9 | 4.5 | 140 | 42 |
| 14 | 20.0 | 4.4 | 100 | 30 |
| 15 | 21.9 | 4.1 | 306 | 92 |
| 16 | 22.1 | 4.0 | 195 | 59 |
| 17 | 22.7 | 3.9 | 91 | 27 |
| 18 | 23.3 | 3.8 | 50 | 15 |
| 19 | 23.4 | 3.8 | 187 | 56 |
| 20 | 23.7 | 3.7 | 181 | 55 |
| 21 | 24.3 | 3.7 | 226 | 68 |
| 22 | 24.6 | 3.6 | 172 | 52 |
| 23 | 25.2 | 3.5 | 263 | 79 |
| 24 | 26.5 | 3.4 | 196 | 59 |
| 25 | 26.7 | 3.3 | 88 | 27 |
| 26 | 27.8 | 3.2 | 114 | 34 |
| 27 | 28.2 | 3.2 | 42 | 13 |
| 28 | 28.5 | 3.1 | 37 | 11 |
| 29 | 29.1 | 3.1 | 94 | 28 |
| 30 | 29.7 | 3.0 | 98 | 30 |
| 31 | 29.9 | 3.0 | 92 | 28 |
| 32 | 30.1 | 3.0 | 99 | 30 |
| 33 | 32.5 | 2.8 | 60 | 18 |
| 34 | 33.6 | 2.7 | 37 | 11 |
| 35 | 35.0 | 2.6 | 56 | 17 |
| 36 | 35.3 | 2.5 | 75 | 23 |
| 37 | 35.8 | 2.5 | 53 | 16 |
| 38 | 37.7 | 2.4 | 121 | 37 |
| 39 | 39.3 | 2.3 | 103 | 31 |

^{a}Bold: Unique set of XRPD Peaks for Form P.
^{b}Intensity of peak/Intensity of most intense peak × 100

DSC

DSC analysis of tiagabine HCl Form P containing a minor amount of Form B obtained by Preparation Method 2 indicated endotherms at 164° C. and 195° C. (major).

Stability

A sample of tiagabine HCl Form P containing a minor amount of Form B obtained by Preparation Method 2 was dried for about 15 hours under vacuum at 40-95° C. XRPD analysis of the resulting sample indicated a mixture of tiagabine HCl Forms P and B.

Samples of tiagabine HCl Form P containing a minor amount of Form B obtained by Preparation Method 2 and 4 were dried for about 4 days under vacuum at room temperature. XRPD analysis of the resulting sample indicated a mixture of tiagabine HCl Forms P and B.

A sample of tiagabine HCl Form P obtained by Preparation Method 1 was stored for five (5) days to about 60° C. and about 75% relative humidity. XRPD analysis of the resulting off-white small needles indicated tiagabine HCl Form B.

A sample of tiagabine HCl Form P obtained by Preparation Method I was stored for five (5) days to about 40° C. and about 89% relative humidity. XRPD analysis of the resulting off-white small needles indicated tiagabine HCl Form B.

A sample of tiagabine HCl Form P containing Form B obtained by Preparation Method 4 was stored for five (5) days at 2-8° C. and about 96% relative humidity. XRPD analysis of the resulting white small needles indicated a mixture of tiagabine HCl Forms P and B.

A sample of tiagabine HCl Form P containing Form B obtained by Preparation Method 3 was dried for about 14 hours under vacuum at about 65° C. XRPD analysis indicated a mixture of tiagabine Form P and B.

### Example 8

### Preparation and Characterization of Tiagabine Hydrochloride Form Q

Preparation Method 1

A mixture of 28 mg of tiagabine HCl amorphous and 2 mL of methyl t-butyl ether was slurried for at room temperature 1 day. The liquid was decanted and the remaining white solids were dried under a gentle stream of nitrogen.

Preparation Method 2

A small amount of tiagabine HCl (Form H) was dried in a vacuum oven at room temperature for 4 days.

Preparation Method 3

Tiagabine HCl monohydrate (130 mg) was dissolved in methanol (250 μL) and refrigerated for 5 days. The solution was removed from the refrigerator and the solvent was evaporated under ambient conditions. The resulting glassy residue was treated with methanol (100 μL), capped, covered with Parafilm®, and slurried for 7 days during which time solids formed.

XRPD

A representative XRPD pattern of tiagabine hydrochloride Form Q is presented in FIG. 8. Representative peaks are listed in the following Table 9.

TABLE 9
Tiagabine HCl Form Q XRPD Peaks

| Peak No.^{a} | Position (°2θ) | d-spacing | Intensity | I/I₀^{b} |
|---|---|---|---|---|
| 1 | 6.4 | 13.9 | 148 | 21 |
| 2 | 11.4 | 7.8 | 95 | 13 |
| 3 | 12.9 | 6.9 | 301 | 42 |
| 4 | 13.5 | 6.5 | 168 | 24 |
| 5 | 13.8 | 6.4 | 145 | 20 |
| 6 | 14.8 | 6.0 | 467 | 66 |
| 7 | 15.3 | 5.8 | 712 | 100 |
| 8 | 16.2 | 5.5 | 155 | 22 |
| 9 | 16.7 | 5.3 | 350 | 49 |
| 10 | 18.3 | 4.8 | 56 | 8 |
| 11 | 18.8 | 4.7 | 202 | 28 |
| 12 | 19.2 | 4.6 | 232 | 33 |
| 13 | 20.9 | 4.2 | 67 | 9 |
| 14 | 22.4 | 4.0 | 68 | 10 |
| 15 | 22.7 | 3.9 | 116 | 16 |
| 16 | 22.9 | 3.9 | 293 | 41 |
| 17 | 23.6 | 3.8 | 83 | 12 |
| 18 | 23.9 | 3.7 | 138 | 19 |
| 19 | 24.4 | 3.6 | 86 | 12 |
| 20 | 24.7 | 3.6 | 376 | 53 |
| 21 | 24.9 | 3.6 | 243 | 34 |
| 22 | 25.3 | 3.5 | 165 | 23 |
| 23 | 26.0 | 3.4 | 107 | 15 |
| 24 | 26.9 | 3.3 | 93 | 13 |

^{a}Bold: Unique set of XRPD Peaks for Form Q
^{b}Intensity of peak/Intensity of most intense peak × 100

TGA

TGA analysis indicated a 1.5% weight loss between 25 to 150° C.

### Example 9

### Preparation and Characterization of Tiagabine Hydrochloride Form T

Approximately 99 mg of tiagabine HCl monohydrate was dissolved in approximately 3 mL of 2-butanol. A clear solution was observed at first and solid quickly precipitated out. The sample vial was capped and slurried at room temperature for 3 days. The resulting solids were collected by filtration and dried in the air.

XRPD

A representative XRPD pattern of tiagabine hydrochloride Form T is presented in FIG. 9. Representative peaks are listed in the following Table 10.

TABLE 10
Tiagabine HCl Form T XRPD Peaks

| Peak No.^{a} | Position (°2θ) | d-spacing | Intensity | I/I₀^{b} |
|---|---|---|---|---|
| 1 | 7.9 | 11.2 | 123 | 77 |
| 2 | 8.6 | 10.2 | 79 | 49 |
| 3 | 11.0 | 8.0 | 32 | 20 |
| 4 | 12.6 | 7.0 | 70 | 44 |
| 5 | 13.8 | 6.4 | 9 | 6 |
| 6 | 14.7 | 6.0 | 36 | 23 |
| 7 | 15.5 | 5.7 | 45 | 28 |
| 8 | 15.9 | 5.6 | 160 | 100 |
| 9 | 16.6 | 5.3 | 22 | 14 |
| 10 | 17.1 | 5.2 | 123 | 77 |
| 11 | 17.3 | 5.1 | 81 | 51 |
| 12 | 17.7 | 5.0 | 22 | 14 |
| 13 | 18.3 | 4.8 | 108 | 68 |
| 14 | 19.2 | 4.6 | 47 | 29 |
| 15 | 19.9 | 4.5 | 24 | 15 |
| 16 | 20.2 | 4.4 | 27 | 17 |
| 17 | 20.8 | 4.3 | 53 | 33 |
| 18 | 21.5 | 4.1 | 46 | 29 |
| 19 | 22.2 | 4.0 | 68 | 43 |
| 20 | 23.0 | 3.9 | 132 | 83 |
| 21 | 23.5 | 3.8 | 130 | 81 |
| 22 | 23.9 | 3.7 | 117 | 73 |
| 23 | 25.0 | 3.6 | 92 | 57 |
| 24 | 25.4 | 3.5 | 57 | 36 |
| 25 | 26.1 | 3.4 | 59 | 37 |
| 26 | 27.1 | 3.3 | 30 | 19 |
| 27 | 27.7 | 3.2 | 30 | 19 |
| 28 | 28.1 | 3.2 | 23 | 14 |
| 29 | 29.4 | 3.0 | 35 | 22 |
| 30 | 30.5 | 2.9 | 17 | 11 |
| 31 | 31.0 | 2.9 | 24 | 15 |
| 32 | 32.0 | 2.8 | 24 | 15 |
| 33 | 32.6 | 2.7 | 31 | 19 |
| 34 | 33.6 | 2.7 | 34 | 21 |
| 35 | 34.1 | 2.6 | 32 | 20 |
| 36 | 37.1 | 2.4 | 54 | 34 |
| 37 | 37.4 | 2.4 | 25 | 16 |
| 38 | 38.4 | 2.3 | 22 | 14 |
| 39 | 39.2 | 2.3 | 30 | 19 |

^{a}Bold: Unique set of XRPD Peaks for Form T
^{b}Intensity of peak/Intensity of most intense peak × 100

TGA

TGA analysis indicated a 7.3% weight loss between 25 to 150° C.

¹H NMR

¹H NMR analysis indicated that the tiagabine hydrochloride Form T contained 0.38 moles of 2-butanol per mole of tiagabine HCl.

Stability

A sample of tiagabine HCl Form T was heated to about 115-120° C. for approximately 10 minutes. XRPD analysis indicated tiagabine HCl Form B.

A sample of tiagabine HCl Form T was dried under vacuum for about 4 days at room temperature. XRPD analysis indicated tiagabine HCl Form C.

### Example 10

### Preparation and Characterization of Tiagabine Hydrochloride Form W

Preparation Method I

A mixture of 115 mg of tiagabine HCl monohydrate and 10 mL of acetone was heated at reflux for about 5 minutes to give a saturated solution. The remaining solids were removed by filtration. The filtrate was collected and cooled in an ice/water bath for about 1 hour. A white precipitate was formed. The liquid was decanted and the remaining white solids were allowed to air dry.

Preparation Method 2

A mixture of 124 mg of tiagabine HCl monohydrate and 10 mL of acetone was heated at reflux for about 5 minutes to give a saturated solution. The remaining solids were removed by filtration. The filtrate was collected and 10 mL of heptane was added. A white precipitate formed. The liquid was decanted and the remaining white solids were allowed to air dry. XRPD analysis indicated a mixture of Form W and Form B.

Preparation Method 3

Tiagabine HCl monohydrate (116 mg) was mixed with acetone (10 mL). Cyclohexane (10 mL) was added. White solids were collected by decantation.

Preparation Method 4

A small amount of tiagabine HCl Form W from Preparation Method 3 was dried under vacuum at room temperature for less than one day.

XRPD

A representative XRPD pattern of tiagabine hydrochloride Form W is presented in FIG. 10. Representative peaks are listed in the following Table 11.

TABLE 11
Tiagabine HCl Form W XRPD Peaks

| Peak No.^{a} | Position (°2θ) | d-spacing | Intensity | I/I₀^{b} |
|---|---|---|---|---|
| 1 | 12.1 | 7.3 | 66 | 12 |
| 2 | 12.6 | 7.0 | 407 | 75 |
| 3 | 13.2 | 6.7 | 393 | 73 |
| 4 | 13.4 | 6.6 | 168 | 31 |
| 5 | 13.9 | 6.3 | 55 | 10 |
| 6 | 14.5 | 6.1 | 36 | 7 |
| 7 | 14.9 | 5.9 | 36 | 7 |
| 8 | 15.2 | 5.8 | 115 | 21 |
| 9 | 16.6 | 5.4 | 541 | 100 |
| 10 | 17.0 | 5.2 | 280 | 52 |
| 11 | 17.6 | 5.0 | 180 | 33 |
| 12 | 18.0 | 4.9 | 104 | 19 |
| 13 | 18.6 | 4.8 | 239 | 44 |
| 14 | 19.8 | 4.5 | 117 | 22 |
| 15 | 20.3 | 4.4 | 42 | 8 |
| 16 | 20.7 | 4.3 | 57 | 11 |
| 17 | 21.0 | 4.2 | 380 | 70 |
| 18 | 21.6 | 4.1 | 116 | 21 |
| 19 | 22.7 | 3.9 | 47 | 9 |
| 20 | 23.0 | 3.9 | 128 | 24 |
| 21 | 23.5 | 3.8 | 50 | 9 |
| 22 | 23.9 | 3.7 | 303 | 56 |
| 23 | 24.3 | 3.7 | 253 | 47 |
| 24 | 24.8 | 3.6 | 420 | 78 |
| 25 | 25.4 | 3.5 | 167 | 31 |
| 26 | 26.4 | 3.4 | 203 | 38 |
| 27 | 27.1 | 3.3 | 46 | 9 |
| 28 | 27.4 | 3.2 | 104 | 19 |
| 29 | 28.2 | 3.2 | 185 | 34 |
| 30 | 28.4 | 3.1 | 75 | 14 |
| 31 | 31.7 | 2.8 | 51 | 9 |
| 32 | 32.7 | 2.7 | 49 | 9 |
| 33 | 33.5 | 2.7 | 74 | 14 |
| 34 | 36.6 | 2.5 | 56 | 10 |

^{a}Bold: Unique set of XRPD Peaks for Form W
^{b}Intensity of peak/Intensity of most intense peak × 100

### Example 11

### Preparation and Characterization of Tiagabine Hydrochloride Form Y

Approximately 27 mg of tiagabine HCl amorphous was dissolved in approximately 0.05 mL of 1,4-dioxane. A clear solution was obtained at first and solids quickly precipitated out. The solvent was removed under a gentle stream of nitrogen and a solid was obtained.

XRPD

A representative XRPD pattern of tiagabine hydrochloride Form Y is presented in FIG. 11. Representative peaks are listed in the following Table 12.

TABLE 12
Tiagabine HCl Form Y XRPD Peaks

| Peak No.^{a} | Position (°2θ) | d-spacing | Intensity | I/I₀^{b} |
|---|---|---|---|---|
| 1 | 5.8 | 15.3 | 142 | 14 |
| 2 | 7.7 | 11.5 | 849 | 85 |
| 3 | 11.6 | 7.6 | 303 | 30 |
| 4 | 14.6 | 6.1 | 905 | 91 |
| 5 | 15.3 | 5.8 | 254 | 25 |
| 6 | 15.5 | 5.7 | 285 | 29 |
| 7 | 16.0 | 5.6 | 131 | 13 |
| 8 | 16.3 | 5.4 | 132 | 13 |
| 9 | 16.7 | 5.3 | 816 | 82 |
| 10 | 16.9 | 5.2 | 998 | 100 |
| 11 | 18.2 | 4.9 | 170 | 17 |
| 12 | 18.6 | 4.8 | 346 | 35 |
| 13 | 18.9 | 4.7 | 325 | 33 |
| 14 | 19.3 | 4.6 | 120 | 12 |
| 15 | 19.7 | 4.5 | 339 | 34 |
| 16 | 19.9 | 4.5 | 165 | 17 |
| 17 | 21.4 | 4.2 | 357 | 36 |
| 18 | 21.8 | 4.1 | 298 | 30 |
| 19 | 22.1 | 4.0 | 301 | 30 |
| 20 | 22.4 | 4.0 | 488 | 49 |
| 21 | 25.0 | 3.6 | 324 | 32 |
| 22 | 25.6 | 3.5 | 492 | 49 |
| 23 | 26.1 | 3.4 | 509 | 51 |
| 24 | 26.6 | 3.3 | 185 | 19 |
| 25 | 27.4 | 3.3 | 323 | 32 |
| 26 | 28.7 | 3.1 | 104 | 10 |
| 27 | 29.9 | 3.0 | 119 | 12 |

^{a}Bold: Unique set of XRPD Peaks for Form Y
^{b}Intensity of peak/Intensity of most intense peak × 100

TGA

TGA analysis indicated a 16.9% weight loss between 25 to 125° C.

¹H NMR

¹H NMR analysis indicated that the tiagabine hydrochloride Form Y contained 0.92 moles of dioxane per mole of tiagabine HCl.

Stability

A sample of tiagabine HCl Form Y was dried under vacuum at room temperature for less than one day. XRPD analysis indicated Form Y.

A sample of tiagabine HCl Form Y was heated at 100-110° C. for about 10 minutes. XRPD analysis of the off-white solids indicated Form B containing a minor amount of Form Q.

### Example 12

### Preparation and Characterization of Tiagabine Hydrochloride Form Z

Approximately 28 mg of tiagabine HCl amorphous was dissolved in approximately 0.05 mL of THF. A clear solution was obtained at first and solids quickly precipitated out. The solvent was removed under a gentle stream of nitrogen and a solid was obtained.

XRPD

A representative XRPD pattern of tiagabine hydrochloride Form Z is presented in FIG. 12. Representative peaks are listed in the following Table 13.

TABLE 13
Tiagabine HCl Form Z XRPD Peaks

| Peak No.^{a} | Position (°2θ) | d-spacing | Intensity | I/I₀^{b} |
|---|---|---|---|---|
| 1 | 5.6 | 15.7 | 434 | 42 |
| 2 | 8.3 | 10.6 | 138 | 13 |
| 3 | 11.4 | 7.8 | 156 | 15 |
| 4 | 11.7 | 7.5 | 182 | 18 |
| 5 | 13.2 | 6.7 | 291 | 28 |
| 6 | 14.6 | 6.1 | 126 | 12 |
| 7 | 16.4 | 5.4 | 1031 | 100 |
| 8 | 16.9 | 5.2 | 603 | 58 |
| 9 | 19.9 | 4.4 | 331 | 32 |
| 10 | 20.3 | 4.4 | 160 | 16 |
| 11 | 20.7 | 4.3 | 321 | 31 |
| 12 | 21.1 | 4.2 | 95 | 9 |
| 13 | 21.5 | 4.1 | 67 | 6 |
| 14 | 22.6 | 3.9 | 179 | 17 |
| 15 | 23.0 | 3.9 | 214 | 21 |
| 16 | 23.6 | 3.8 | 116 | 11 |
| 17 | 23.9 | 3.7 | 588 | 57 |
| 18 | 24.3 | 3.7 | 369 | 36 |
| 19 | 24.6 | 3.6 | 376 | 36 |
| 20 | 25.5 | 3.5 | 323 | 31 |
| 21 | 25.9 | 3.4 | 123 | 12 |
| 22 | 26.6 | 3.4 | 88 | 9 |
| 23 | 26.9 | 3.3 | 44 | 4 |
| 24 | 28.1 | 3.2 | 35 | 3 |
| 25 | 28.9 | 3.1 | 84 | 8 |
| 26 | 29.4 | 3.0 | 78 | 8 |
| 27 | 30.7 | 2.9 | 127 | 12 |
| 28 | 32.3 | 2.8 | 69 | 7 |
| 29 | 33.2 | 2.7 | 54 | 5 |
| 30 | 34.2 | 2.6 | 86 | 8 |

^{a}Bold: Unique set of XRPD Peaks for Form Z
^{b}Intensity of peak/Intensity of most intense peak × 100

TGA

TGA analysis indicated a 13.0% weight loss between 25 to 100° C.

¹H NMR

¹H NMR analysis indicated that the tiagabine hydrochloride Form Z contained 0.59 moles of tetrahydrofuran per mole of tiagabine HCl.

Stability

From Z obtained by Example 12 was heated at 90 to 95° C. for approximately 10 minutes. XRPD analysis indicated a mixture of Forms B and Q.

### Example 13

### Preparation and Characterization of Tiagabine Hydrochloride Form AA

A mixture 114 mg of tiagabine HCl monohydrate and 4 mL of acetone was slurried for 4 days at room temperature. The white solids were collected by filtration and air dried.

Stability

A sample of tiagabine HCl Form AA obtained was dried under vacuum at room temperature for less than one day. XRPD analysis indicated Form AA.

XRPD

A representative XRPD pattern of tiagabine hydrochloride Form AA is presented in FIG. 13. Representative peaks are listed in the following Table 14.

TABLE 14
Tiagabine HCl Form AA XRPD Peaks

| Peak No.^{a} | Position (°2θ) | d-spacing | Intensity | I/I₀^{b} |
|---|---|---|---|---|
| 1 | 7.4 | 12.0 | 31 | 30 |
| 2 | 11.2 | 7.9 | 56 | 54 |
| 3 | 12.1 | 7.3 | 22 | 21 |
| 4 | 12.5 | 7.1 | 17 | 17 |
| 5 | 12.8 | 6.9 | 50 | 49 |
| 6 | 13.1 | 6.8 | 61 | 59 |
| 7 | 13.5 | 6.6 | 12 | 12 |
| 8 | 13.7 | 6.5 | 33 | 32 |
| 9 | 13.9 | 6.4 | 39 | 38 |
| 10 | 14.7 | 6.0 | 67 | 65 |
| 11 | 16.6 | 5.3 | 71 | 69 |
| 12 | 17.1 | 5.2 | 12 | 12 |
| 13 | 17.3 | 5.1 | 23 | 22 |
| 14 | 18.0 | 4.9 | 12 | 12 |
| 15 | 18.2 | 4.9 | 103 | 100 |
| 16 | 20.0 | 4.4 | 63 | 61 |
| 17 | 20.7 | 4.3 | 22 | 21 |
| 18 | 21.1 | 4.2 | 20 | 19 |
| 19 | 21.3 | 4.2 | 18 | 17 |
| 20 | 21.7 | 4.1 | 49 | 48 |
| 21 | 22.0 | 4.0 | 57 | 55 |
| 22 | 22.4 | 4.0 | 48 | 47 |
| 23 | 23.2 | 3.8 | 12 | 12 |
| 24 | 23.7 | 3.8 | 35 | 34 |
| 25 | 24.0 | 3.7 | 71 | 69 |
| 26 | 24.3 | 3.7 | 56 | 54 |
| 27 | 25.4 | 3.5 | 45 | 44 |
| 28 | 25.6 | 3.5 | 42 | 41 |
| 29 | 26.8 | 3.3 | 30 | 29 |
| 30 | 27.5 | 3.2 | 33 | 32 |
| 31 | 28.7 | 3.1 | 17 | 17 |
| 32 | 29.9 | 3.0 | 24 | 23 |
| 33 | 30.6 | 2.9 | 21 | 20 |
| 34 | 32.4 | 2.8 | 13 | 13 |
| 35 | 33.1 | 2.7 | 30 | 29 |
| 36 | 34.6 | 2.6 | 27 | 26 |
| 37 | 35.9 | 2.5 | 29 | 28 |

^{a}Bold: Unique set of XRPD Peaks for Form AA
^{b}Intensity of peak/Intensity of most intense peak × 100

### Example 14

### Preparation and Characterization of Tiagabine Hydrochloride Form S

Preparation Method 1

Tiagabine hydrochloride Form I was stored at room temperature for about two months. XRPD analysis indicated a mixture of Form S+Form B.

Preparation Method 2

A small portion of the tiagabine HCl Form S+B mixture from Preparation Method 1 was placed in a vacuum oven and heated from room temperature to 65° C. for less than one day.

XRPD

XRPD analysis indicated that tiagabine hydrochloride Form S was obtained as a mixture with Form B. A representative XRPD pattern of tiagabine hydrochloride Form S mixture with Form B is presented in FIG. 14. Representative peaks are listed in the following Table 15.

TABLE 15
Tiagabine HCl Form S XRPD Peaks

| Peak No. | Position (°2θ) | d-spacing | Intensity | I/I₀ |
|---|---|---|---|---|
| 1 | 6.4 | 13.8 | 111 | 14 |
| 2 | 6.7 | 13.1 | 229 | 29 |
| 3 | 7.9 | 11.2 | 508 | 64 |
| 4 | 11.3 | 7.8 | 84 | 11 |
| 5 | 12.5 | 7.1 | 611 | 77 |
| 6 | 12.9 | 6.9 | 129 | 16 |
| 7 | 13.1 | 6.7 | 127 | 16 |
| 8 | 13.5 | 6.5 | 250 | 31 |
| 9 | 14.3 | 6.2 | 276 | 35 |
| 10 | 14.8 | 6.0 | 222 | 28 |
| 11 | 15.3 | 5.8 | 121 | 15 |
| 12 | 15.4 | 5.7 | 155 | 19 |
| 13 | 15.9 | 5.6 | 545 | 68 |
| 14 | 16.2 | 5.5 | 86 | 11 |
| 15 | 16.7 | 5.3 | 146 | 18 |
| 16 | 17.6 | 5.0 | 229 | 29 |
| 17 | 18.3 | 4.8 | 264 | 33 |
| 18 | 18.5 | 4.8 | 251 | 32 |
| 19 | 19.3 | 4.6 | 80 | 10 |
| 20 | 19.6 | 4.5 | 149 | 19 |
| 21 | 19.9 | 4.5 | 126 | 16 |
| 22 | 20.3 | 4.4 | 128 | 16 |
| 23 | 20.9 | 4.2 | 64 | 8 |
| 24 | 21.8 | 4.1 | 375 | 47 |
| 25 | 22.6 | 3.9 | 126 | 16 |
| 26 | 23.5 | 3.8 | 140 | 18 |
| 27 | 23.7 | 3.7 | 194 | 24 |
| 28 | 24.8 | 3.6 | 796 | 100 |
| 29 | 25.2 | 3.5 | 291 | 37 |
| 30 | 25.6 | 3.5 | 79 | 10 |
| 31 | 26.1 | 3.4 | 80 | 10 |
| 32 | 26.5 | 3.4 | 242 | 30 |
| 33 | 27.3 | 3.3 | 154 | 19 |
| 34 | 27.7 | 3.2 | 159 | 20 |
| 35 | 28.8 | 3.1 | 72 | 9 |
| 36 | 29.6 | 3.0 | 136 | 17 |
| 37 | 30.0 | 3.0 | 62 | 8 |
| 38 | 30.4 | 2.9 | 76 | 10 |
| 39 | 32.5 | 2.8 | 99 | 12 |
| 40 | 35.1 | 2.6 | 67 | 8 |
| 41 | 35.4 | 2.5 | 90 | 11 |
| 42 | 36.5 | 2.5 | 75 | 9 |
| 43 | 37.7 | 2.4 | 100 | 13 |
| 44 | 38.4 | 2.3 | 65 | 8 |
| 45 | 39.8 | 2.3 | 80 | 10 |

^{a}Bold: Unique set of XRPD Peaks for Form S
^{b}Intensity of peak/Intensity of most intense peak × 100

DSC

DSC analysis indicated endotherms at 126° C. and 197° C. (major). A representative DSC curve of Form S mixture with Form B is presented in FIG. 15.

TGA

TGA analysis indicated a 2.8% weight loss between 25 to 150° C.

¹H NMR

¹H NMR analysis indicated that the mixture of Form S and Form B contained 0.17 moles of acetone per mole of tiagabine hydrochloride.

Stability

A sample of tiagabine HCl Form S mixed with Form B was dried for about 14 hours under vacuum at about 65° C. XRPD analysis of the resulting sample indicated a mixture of tiagabine HCl Forms S and B.

### Example 15

### Preparation and Characterization of Tiagabine Hydrochloride Form X

Approximately 10 mg of tiagabine HCl amorphous was dissolved in approximately 0.02 mL of water. A clear solution was obtained at first and solids quickly precipitated out. The solvent was evaporated in the opened vial to give a white, needle, solid.

XRPD

XRPD analysis indicated that tiagabine hydrochloride Form X was obtained as a mixture with Form A. A representative XRPD pattern of tiagabine hydrochloride Form X mixture with Form A is presented in FIG. 16. Representative peaks are listed in the following Table 16.

TABLE 16
Tiagabine HCl Form X XRPD Peaks

| Peak No.^{a} | Position (°2θ) | d-spacing | Intensity | I/I₀^{b} |
|---|---|---|---|---|
| 1 | 7.3 | 12.1 | 270 | 50 |
| 2 | 7.8 | 11.3 | 231 | 43 |
| 3 | 11.7 | 7.5 | 374 | 70 |
| 4 | 12.1 | 7.3 | 129 | 24 |
| 5 | 12.8 | 6.9 | 192 | 36 |
| 6 | 13.1 | 6.7 | 309 | 58 |
| 7 | 13.6 | 6.5 | 196 | 37 |
| 8 | 14.0 | 6.3 | 292 | 54 |
| 9 | 14.5 | 6.1 | 119 | 22 |
| 10 | 14.7 | 6.0 | 238 | 44 |
| 11 | 15.6 | 5.7 | 302 | 56 |
| 12 | 16.6 | 5.3 | 221 | 41 |
| 13 | 16.9 | 5.2 | 180 | 34 |
| 14 | 17.7 | 5.0 | 317 | 59 |
| 15 | 18.1 | 4.9 | 364 | 68 |
| 16 | 18.5 | 4.8 | 154 | 29 |
| 17 | 18.9 | 4.7 | 166 | 31 |
| 18 | 19.6 | 4.5 | 142 | 26 |
| 19 | 20.0 | 4.4 | 270 | 50 |
| 20 | 20.8 | 4.3 | 125 | 23 |
| 21 | 21.5 | 4.1 | 150 | 28 |
| 22 | 21.9 | 4.1 | 285 | 53 |
| 23 | 22.3 | 4.0 | 350 | 65 |
| 24 | 23.2 | 3.8 | 143 | 27 |
| 25 | 23.4 | 3.8 | 101 | 19 |
| 26 | 23.8 | 3.7 | 167 | 31 |
| 27 | 24.4 | 3.6 | 345 | 64 |
| 28 | 24.9 | 3.6 | 382 | 71 |
| 29 | 25.4 | 3.5 | 536 | 100 |
| 30 | 25.6 | 3.5 | 283 | 53 |
| 31 | 25.8 | 3.4 | 184 | 34 |
| 32 | 26.3 | 3.4 | 221 | 41 |
| 33 | 26.7 | 3.3 | 305 | 57 |
| 34 | 27.0 | 3.3 | 106 | 20 |
| 35 | 27.4 | 3.2 | 199 | 37 |
| 36 | 27.9 | 3.2 | 156 | 29 |
| 37 | 28.6 | 3.1 | 149 | 28 |
| 38 | 28.8 | 3.1 | 112 | 21 |
| 39 | 29.2 | 3.1 | 103 | 19 |
| 40 | 29.4 | 3.0 | 106 | 20 |
| 41 | 30.1 | 3.0 | 161 | 30 |
| 42 | 33.3 | 2.7 | 94 | 18 |
| 43 | 35.9 | 2.5 | 93 | 17 |

^{a}Bold: Unique set of XRPD Peaks for Form X
^{b}Intensity of peak/Intensity of most intense peak × 100

### Example 16

### Preparation and Characterization of Tiagabine Hydrochloride Form AB

Approximately 501 mg of tiagabine HCl monohydrate was heated at 150° C. under nitrogen atmosphere for about 10 minutes. It was observed that some solids on the bottom were partially melted. The sample was then stored under subambient conditions in a desiccator containing phosphorus pentoxide.

XRPD

XRPD analysis indicated that tiagabine hydrochloride Form AB was obtained as a mixture with Form B. A representative XRPD pattern of tiagabine hydrochloride Form AB mixture with Form B is presented in FIG. 17. Representative peaks are listed in the following Table 17.

TABLE 17
Tiagabine HCl Form AB XRPD Peaks

| Peak No.^{a} | Position (°2θ) | d-spacing | Intensity | I/Iₒ^{b} |
|---|---|---|---|---|
| 1 | 4.1 | 21.3 | 101 | 16 |
| 2 | 6.4 | 13.7 | 89 | 14 |
| 3 | 7.6 | 11.6 | 457 | 70 |
| 4 | 11.3 | 7.8 | 98 | 15 |
| 5 | 12.5 | 7.1 | 150 | 23 |
| 6 | 12.8 | 6.9 | 255 | 39 |
| 7 | 13.4 | 6.6 | 560 | 86 |
| 8 | 14.0 | 6.3 | 136 | 21 |
| 9 | 14.5 | 6.1 | 141 | 22 |
| 10 | 14.8 | 6.0 | 424 | 65 |
| 11 | 15.2 | 5.8 | 168 | 26 |
| 12 | 15.9 | 5.6 | 136 | 21 |
| 13 | 16.1 | 5.5 | 102 | 16 |
| 14 | 16.6 | 5.3 | 284 | 44 |
| 15 | 17.8 | 5.0 | 175 | 27 |
| 16 | 18.4 | 4.8 | 650 | 100 |
| 17 | 19.4 | 4.6 | 140 | 22 |
| 18 | 19.9 | 4.5 | 166 | 26 |
| 19 | 20.3 | 4.4 | 253 | 39 |
| 20 | 20.9 | 4.2 | 163 | 25 |
| 21 | 21.8 | 4.1 | 101 | 16 |
| 22 | 22.3 | 4.0 | 120 | 18 |
| 23 | 22.6 | 3.9 | 243 | 37 |
| 24 | 23.3 | 3.8 | 150 | 23 |
| 25 | 23.6 | 3.8 | 113 | 17 |
| 26 | 23.9 | 3.7 | 82 | 13 |
| 27 | 24.8 | 3.6 | 438 | 67 |
| 28 | 25.2 | 3.5 | 206 | 32 |
| 29 | 25.6 | 3.5 | 199 | 31 |
| 30 | 25.9 | 3.4 | 232 | 36 |
| 31 | 26.3 | 3.4 | 79 | 12 |
| 32 | 27.0 | 3.3 | 117 | 18 |
| 33 | 27.3 | 3.3 | 160 | 25 |
| 34 | 28.3 | 3.2 | 77 | 12 |
| 35 | 29.0 | 3.1 | 116 | 18 |
| 36 | 29.9 | 3.0 | 74 | 11 |
| 37 | 34.4 | 2.6 | 62 | 10 |

^{a}Bold: Unique set of XRPD Peaks for Form AB
^{b}Intensity of peak/Intensity of most intense peak × 100

### Example 17

### Preparation and Characterization of Tiagabine Hydrochloride Amorphous

Preparation Method 1

0.1 g of tiagabine HCl was placed in a vial. The sample was heated at 204° C. in an oil bath under vacuum for about 5 minutes. The sample was completely melted. The sample was then crash-cooled by immersing in an ice bath. The glassy solids were ground in a mortar into small plates before analysis. The obtained product was amorphous, composed of small plates, and without birefringence.

Preparation Method 2

0.1 g of tiagabine HCl was placed in a vial. The sample was placed under a gentle nitrogen stream and then heated at 200° C. in an oil bath for one minute. The sample was completely melted. The sample was heated in the bath for an additional 3 minutes before it was immersed in a dry ice/isopropanol bath. The obtained product was amorphous, brown/dark yellow in color, glassy, and without birefringence.

Preparation Method 3

0.2 g of tiagabine HCl was dissolved in 20 mL of water to give a clear solution. The solution was filtered through a 0.2 μm filter. The filtrate was frozen in a dry ice/acetone bath, and then dried in a freeze dryer under high vacuum.

Preparation Method 4

Tiagabine HCl form B (32 mg) was placed in a grinding jar with a 5 mm stainless steel ball. The sample was milled for 10 minute intervals (3×10 minutes=30 minutes) at 30 Hz using a Retsch MM200 mixer mill. Solids were scraped from the sides of the vial after each interval. Sample was collected in a vial.

XRPD

A representative XRPD pattern of tiagabine hydrochloride amorphous obtained by Preparation Method 1 is presented in FIG. 18.

A representative XRPD pattern of tiagabine hydrochloride amorphous obtained by Preparation Method 2 is presented in FIG. 19.

A representative XRPD pattern of tiagabine hydrochloride amorphous obtained by Preparation Method 3 is presented in FIG. 20.

DSC

DSC analysis was performed at a heating rate of 11° C./min, up to a final temperature of 250° C. Endotherms were observed at 52, 59, and 189° C., and an exotherm was observed at 152° C. A representative DSC curve of tiagabine HCl amorphous obtained by Preparation Method 1 is presented in FIG. 21.

TGA

TGA analysis indicated a 1.3% weight loss at 95° C.

Moisture Sorption/Desorption

Moisture sorption/desorption analysis indicated an 12.1% weight gain upon sorption at 95% relative humidity (RH), and a 9.5% weight loss upon desorption from 95% to 5% RH. XRPD analysis of the sample after moisture sorption/desorption indicated the presence of tiagabine HCl Forms A and B.

Solubility

The approximate solubility of tiagabine HCl amorphous obtained by Preparation Method 3 in various solvents was determined by adding aliquots (10-25 μL) of a solvent to a weighed sample until complete dissolution was obtained, if possible. Dissolution was determined visually. The actual solubilities may be higher than reported due to the use of excess solvent (e.g., because of slow dissolution rates). The results are presented in the following Table 18.

TABLE 18
Approximate Solubility of Tiagabine HCl Amorphous obtained by
Preparation Method 3

| | Solubility | |
|---|---|---|
| Solvent | (mg/mL) | Observations |
| Acetonitrile | <26 | clear solution then solids precipitated |
| Benzonitrile | >448 | clear solution |
| Chloroform | >582 | clear solution then solids precipitated |
| Dichloromethane | <34 | clear solution, with clumps |
| Dioxane | >542 | clear solution then solids precipitated |
| ethyl acetate | <24 | clear solution then solids precipitated |
| ethyl acetate (wet) | >1190 | Clear solution, possibly oiled out due to emulsion of |
| | | EtOAc and water |
| methyl ethyl ketone | >546 | clear solution then solids precipitated |
| methyl-tert-butyl ether | <14 | cloudy solution, never cleared |
| Tetrahydrofuran | >562 | clear solution then solids precipitated |
| Toluene | <43 | cloudy solution, never cleared |
| Trifluoroethanol | >713 | clear solution then solids precipitated |
| Water | >490 | clear solution; off white needles formed with evaporation |
| 1,4 dioxane/water | >359 | clear solution then solids precipitated; solids dissolved |
| (3:1) | | upon addition of water after a few min; blades and plates |
| | | in purple solution |
| ethanol/water (10:1) | >400 | clear solution; solution turned purple later (with no solids) |
| 2-propanol/water (1:1) | >129 | clear solution; solution turned purple later (with no solids) |

Stability

A sample of tiagabine HCl amorphous obtained by lyophilization as in Preparation Method 3 was heated for five (5) minutes at about 160° C. in an argon atmosphere. XRPD analysis of the resulting blades/plates with foam residue indicated tiagabine HCl Form B.

Two more samples of tiagabine HCl amorphous obtained by lyophilization as in Preparation Method 3 were stored for 5 or 8 days, respectively, at about 5° C. and either about 11% or about 43% relative humidity. XRPD analysis of the resulting samples indicated tiagabine HCl amorphous.

Two more samples of tiagabine HCl amorphous obtained by crash cooling as in Preparation Method 1 were stored for 22 days at room temperature and either about 33% or about 58% relative humidity. XRPD analysis of the resulting samples indicated tiagabine HCl amorphous.

Two more samples of tiagabine HCl amorphous obtained by crash cooling as in Preparation Method 1 were stored for 22 days at room temperature and either about 75% or about 84% relative humidity. XRPD analysis of the resulting samples indicated a mixture of tiagabine hydrochloride Forms A and B.

The citation and discussion of references in this specification is provided merely to clarify the description of the present invention and is not an admission that any such reference is “prior art” to the invention described herein. Each reference cited in this specification is incorporated herein by reference in its entirety.

What is claimed is:

1. A crystalline form of tiagabine hydrochloride chosen from Forms C, D, H, I, J, M, P, Q, T, W, Y, Z, AA, S, X, and AB.

2. The crystalline form of tiagabine hydrochloride of claim 1, wherein the crystalline form is chosen from Forms C, Q, W and AA.

3. The crystalline form of tiagabine hydrochloride of claim 1, wherein the crystalline form exhibits an x-ray powder diffraction pattern having characteristic peaks as set forth in the following table:

| Form | Characteristic XRPD Peaks (±0.2 degrees 2θ) | | | | | | | | | |
|---|---|---|---|---|---|---|---|---|---|---|
| C | 6.1 | 7.9 | 8.7 | 12.7 | 14.8 | 16.1 | 17.2 | 22.9 | 25.1 | 25.9 |
| D | 7.9 | 12.7 | 14.4 | 16.9 | 17.1 | 18.1 | 18.8 | 21.5 | 22.0 | 24.3 |
| H | 5.8 | 7.6 | 7.8 | 11.6 | 14.6 | 15.9 | 17.0 | 19.7 | 22.6 | 25.1 |
| I | 10.5 | 12.5 | 13.1 | 15.0 | 17.3 | 20.6 | 21.0 | 24.8 | 25.2 | 27.0 |
| J | 7.8 | 12.4 | 13.0 | 14.6 | 17.0 | 17.5 | 21.1 | 21.8 | 24.8 | 26.2 |
| M | 7.8 | 12.8 | 14.5 | 16.9 | 21.1 | 21.8 | 24.5 | 24.9 | 26.3 | 27.5 |
| P | 12.5 | 14.5 | 16.1 | 17.6 | 21.9 | 25.2 | 26.5 | 35.8 | 37.7 | 39.3 |
| Q | 6.4 | 11.4 | 12.9 | 14.8 | 15.3 | 16.7 | 18.8 | 22.9 | 24.7 | 25.3 |
| T | 7.9 | 8.6 | 12.6 | 15.9 | 17.1 | 18.3 | 20.8 | 22.2 | 23.5 | 25.0 |
| W | 12.6 | 13.2 | 16.6 | 17.0 | 17.6 | 18.6 | 21.0 | 23.9 | 24.3 | 24.8 |
| Y | 7.7 | 11.6 | 14.6 | 16.7 | 16.9 | 18.6 | 18.9 | 21.4 | 22.4 | 25.6 |
| Z | 5.6 | 8.3 | 11.4 | 11.7 | 13.2 | 16.4 | 16.9 | 19.9 | 20.7 | 23.9 |
| AA | 7.4 | 11.2 | 13.1 | 14.7 | 16.6 | 18.2 | 20.0 | 22.0 | 22.4 | 24.0 |
| S | 6.7 | 7.9 | 12.5 | 13.1 | 17.6 | 21.8 | 27.7 | — | — | — |
| X | 7.8 | 11.7 | 14.0 | 15.6 | 18.5 | 18.9 | 24.9 | — | — | — |
| AB | 4.1 | 7.6 | 14.0 | 17.8 | 18.4 | — | — | — | — | — |

4. The crystalline form of tiagabine hydrochloride of claim 3, wherein the crystalline form is chosen from Forms C, Q, W and AA.

5. The crystalline form of claim 1, wherein the crystalline form has a purity of at least about 50% (w/w).

6. The crystalline form of claim 3, wherein the crystalline form has a purity of at least 15 about 50% (w/w).

7. A pharmaceutical composition comprising one or more crystalline forms of tiagabine hydrochloride according to claim 1 and one or more pharmaceutically acceptable excipients.

8. A pharmaceutical composition comprising one or more crystalline forms of tiagabine hydrochloride according to claim 2 and one or more pharmaceutically acceptable excipients.

9. A pharmaceutical composition comprising one or more crystalline forms of tiagabine hydrochloride according to claim 3 and one or more pharmaceutically acceptable excipients.

10. A pharmaceutical composition comprising one or more crystalline forms of tiagabine hydrochloride according to claim 4 and one or more pharmaceutically acceptable excipients.

11. A process for preparing a crystalline form of tiagabine hydrochloride comprising the steps of:
(s) crystallizing tiagabine hydrochloride from isopropanol to provide tiagabine hydrochloride Form C; or
(t) crystallizing tiagabine hydrochloride from acetonitrile to provide tiagabine hydrochloride Form D; or
(u) crystallizing tiagabine hydrochloride from methyl ethyl ketone to provide tiagabine hydrochloride Form H; or
(v) crystallizing tiagabine hydrochloride from acetone to provide tiagabine hydrochloride Form I; or
(w) crystallizing tiagabine hydrochloride from ethanol to provide tiagabine hydrochloride Form J; or
(x) crystallizing tiagabine hydrochloride from dichloromethane to provide tiagabine hydrochloride Form M; or
(y) crystallizing tiagabine hydrochloride from a solvent selected from 1,4-dioxane and methyl ethyl ketone to provide tiagabine hydrochloride Form P; or
(z) crystallizing tiagabine hydrochloride from methyl t-butyl ether to provide tiagabine hydrochloride Form Q; or
(aa) drying tiagabine hydrochloride Form H in a vacuum oven to provide tiagabine hydrochloride Form Q; or
(bb) crystallizing tiagabine hydrochloride from 2-butanol to provide tiagabine hydrochloride Form T; or
(cc) crystallizing tiagabine hydrochloride from acetone to provide tiagabine hydrochloride Form W; or
(dd) crystallizing tiagabine hydrochloride from a mixture of acetone and cyclohexane to provide tiagabine hydrochloride Form W; or
(ee) crystallizing tiagabine hydrochloride from 1,4-dioxane to provide tiagabine hydrochloride Form Y; or
(ff) crystallizing tiagabine hydrochloride from tetrahydrofuran to provide tiagabine hydrochloride Form Z; or
(gg) slurrying tiagabine hydrochloride monohydrate in acetone to provide tiagabine hydrochloride Form AA; or
(hh) storing tiagabine hydrochloride Form I at room temperature for about two (2) months to provide tiagabine hydrochloride Form S; or
(ii) crystallizing tiagabine hydrochloride from water to provide tiagabine hydrochloride Form X; or
(jj) heating tiagabine hydrochloride monohydrate at 150° C. to provide tiagabine hydrochloride Form AB.

12. A process for preparing amorphous tiagabine hydrochloride, comprising the steps of:
(a) heating tiagabine hydrochloride at or above its melting point, and
(b) cooling the heated tiagabine hydrochloride.

13. The process of claim 12, wherein the cooling step (b) is performed by immersing a container of the melted tiagabine hydrochloride in an ice bath.

14. The process of claim 12, wherein the cooling step (b) is performed by immersing a container of the melted tiagabine hydrochloride in a dry ice/isopropanol bath.

15. A process for preparing amorphous tiagabine hydrochloride, comprising the steps of:
(a) preparing an aqueous solution of tiagabine hydrochloride, and
(b) freeze drying the aqueous solution of tiagabine hydrochloride.
